(19) United States
(12) Patent Application Publication
(10) Pub. No.: US 2017/0027347 A1
(43) Pub. Date: Feb. 2, 2017

(54) Disposable Sanitary Tray and Bedside Tables

(71) Applicant: Steven Gabor Kovacs, Parker, CO (US)

(72) Inventor: Steven Gabor Kovacs, Parker, CO (US)

(21) Appl. No.: 15/090,613

(22) Filed: Apr. 4, 2016

Related U.S. Application Data
(60) Provisional application No. 62/142,785, filed on Apr. 3, 2015.

(51) Int. Cl. A47G 19/03; A47G 23/06; B65D 81/26; A61L 2/03; A47B 9/06; A47B 23/04; A61L 2/232; A61L 2/10; B65D 1/36; A47B 13/08

(52) U.S. Cl. CPC A47G 19/03; B65D 1/36; A47G 23/06; B65D 81/264; A47B 13/086; A47B 13/081; A47B 9/06; A47B 23/046; A61L 2/232; A61L 2/10; A61L 2/03; A47G 2023/0658; A47B 2023/047

(57) ABSTRACT

The present invention teaches a bedside table with a disposable hygienic tray which has thereon multiple layers of film. Each layer of film in turn has adhesive, or anti-bacterial compounds, or bacterial detection compounds, or combinations thereof. In use, for example in a hospital, health care setting, home care setting or for home or many other contexts of use, each layer is removed in sequence, exposing a new layer underneath. After a suitable period of usage, the new layer is in turn removed to expose yet another later. When the final layer is removed the disposable tray is removed from the bedside table and replaced with a fresh tray. In embodiments, the core bedside table unit may sit on the floor, mount to a wall rail, or may have a projection dimensioned and configured to pass between the bottom of a mattress and the top of a box spring.

### CROSS-REFERENCE TO RELATED APPLICATIONS

The application claims the priority and benefit of previously filed and presently co-pending U.S. Provisional Application No. 62/142,785 filed on Apr. 3, 2015 in the name of the same inventor Steven Gabor Kovacs and having the same title, the entirety of which is incorporated herein by this reference.

### FIELD OF INVENTION

This invention relates generally to disposable tray tables of the caddy or container type, and in particular to the use of compounds having anti-bacterial/bacteria detection properties on multiple peelable layers of a disposable tray fitted to a bedside/sofaside/chairborne table adapted to the tray and to the bed, as well as diverse accessories.

### STATEMENT REGARDING FEDERALLY FUNDED RESEARCH

This invention was not made under contract with an agency of the US Government, nor by any agency of the US Government.

### BACKGROUND OF THE INVENTION

While sneezing and coughing are commonly imagined to be the leading causes of infection, in fact contact, direct and indirect, are enormous vectors for infection. Common activities like shaking hands, passing beverages or food containers, handing over a credit card or money, or touching a counter-top, table or piece of paper are all extremely common routes for disease transmission, or for that matter on any item which is transferred hand to hand and thus includes touching.

This type of spread of infection occurs in numerous settings: fast food restaurant trays, cafeteria trays, hospital bedside tables and trays (“overbed tables”), hospice and elder care facilities, home care settings and more.

There are at least some data points available in the healthcare industry setting. Approximately 3.7 million persons per year get an infection in the healthcare setting in the US, with approximately half a million deaths resulting, making it the third leading cause of death after cancer and heart disease. The 2011 treatment cost was estimated at over $70 Billion, and beginning in 2012 Medicare and Medicaid began to limit payments of healthcare costs associated with healthcare-system-acquired infections.

Obviously, the corresponding figures for other settings are very difficult to establish but given the efforts at hygiene made in the healthcare setting compared to the efforts elsewhere it seems likely that the true, unknowable, figures are much worse.

There are many efforts made to limit or slow the spread of disease, ranging from the trivial to the extremely efficient. Restaurants and cafeterias are expected to wipe down trays and countertops according to health regulations, hospitals sanitize equipment much more stringently. There are liquid solutions, UV sterilizers, occasional tests for the presence of micro-organisms and so forth. Many of these require enormous amounts of time-intensive labor, others have financial costs.

In the hospital setting, the places where a patient is most likely to stay, be treated, touch or otherwise impact are considered the highest priorities for cleaning. A partial list includes not just patient bedrooms but waiting rooms, operating rooms and so on. Within these rooms, the “high touch” areas fall under particular scrutiny since contact is the number one vector for transmission of disease, not air-borne routes. High touch areas include some places about which nothing can be done but frequent cleaning (television remote controls or bathroom surfaces) but do include some areas which could be handled in less labor intensive ways. In particular, the top, front, sides and rails of beds, head boards, and especially bed side tables, drawers, light switches and the like.

After cleaning, the cleaning person must remove their gloves and outer garments and the like and those must also be cleaned. After patient discharge additional items have to be cleaned.

Studies have shown that methicillin-resistant Staphylococcus aureus (MRSA) and Clostridium difficile are strongly impacted by the type of surface they rest on and the location of the surface (how humans use the surface) is important as well. For example, cork is much more likely to harbor microorganic life than plastic, but any surface may become contaminated with the passage of too much time.

Cleaning protocols in use appear to be inadequate given the high prevalence found in hospital environments of not just the two pathogens above but multi-drug resistant Acinetobacter baumannii (MDR) and others. In one study, MDR could be found in over half the the rooms tested, AFTER they rooms had been cleaned. Once again, the prime suspects list for locations of contamination included parts of the bedside table, supply carts, and so on.

And these studies are in the hospital setting, where the fight against contamination is waged most energetically. It is rather frightening to consider the home or hotel environment where there are no trained personnel available to combat the spread of pathogens.

When one considers that hospital grade cleaning compounds must be inexpensive, easy to apply, and EPA approved disinfectants, and the enormous manual labor involved in cleaning, it becomes clear that better cleaning solutions are necessary.

It would be preferable to provide a clean, sterile surface without having to engage in constant cleaning. It would further be preferable to do so with very low labor cost and using lost cost equipment.

It would yet further be preferable to provide some system which could be used in a wide variety of settings, from the home to the hospital to the airline to restaurants and more.

It would yet further be preferable to provide a furniture system incorporating these elements for use with beds, chairs, couches, and trays regardless of the context of the use.

It would yet further be preferable to provide a furniture system having diverse accessories such as UV lights, electric power, data connections and so on in a hygienic and safe furniture system.

One example of peelable technology for sanitation is the peelable shower curtain shown in US Patent No. 2007/0215296 dated Nov. 1, 1994. However, this curtain is not for handling, it is not for support of food trays, canteens, containers, personal devices or the like, and it lacks any support structures such as a firm backing layer or a table structure, a base, etc. It also lacks any suggestion, or even any reason why a shower curtain might be combined with a bedside table.

U.S. Pat. No. 7,997,211 and US Patent Applications 2004/01748312, 2005/0022699, and 2005/0045788, and 2008/0006183 all show various types of arm arrangements for hospital rooms and the like, of various rather general natures.

Thus it would be further preferable to provide a method of using peelable technology combined with table/TV trays/night stands/end tables/coffee tables and tray furnishings, canteens, cups, containers of many types, personal devices and so on and so forth.

### SUMMARY OF THE INVENTION

A compact environmentally transforming platform that connects to a cartridge/tray providing a peel-away controllable film surface eliminating and or detecting the presence of a threatening antibiotic resistant pathogen and or dangerous bacteria.

The present invention teaches a bedside table with a disposable hygienic tray which has thereon multiple layers of film. In embodiments, the tray may be designed to fit on a standard bedside table, however in the best mode now contemplated the tray fits onto a specially designed pair of tongs, prongs or forks projecting from a specially designed table. The fork can provide low voltage or data to the tray frame and to the film layers. UV lighting or the like may also be provided, as well as conventional reading light or night lights.

Each layer of film in has adhesive, or anti-bacterial compounds, or bacterial detection compounds, or combinations thereof. For example, in an embodiment in which static and/or suction cause the layers to cling to one another, each layer might have thereon a thin coating of a compound which kills bacteria or fungus or viruses or prions or other sources of infection. In another embodiment, there might be stripes or patterns of one material side by side with stripes or patterns of another, while in yet a third embodiment there might be alternating layers, or the adhesive may be combined with another compound, etc. In yet another embodiment, the film may have low voltage (and may have the proper conductivity characteristics therefor) flowing through it: this may activate the compounds of the film or the compounds between the films, or may have beneficial characteristics of its own. The compounds may be time release types as well as liquids and solids.

Films may have texturing or microchannels to enhance the flow of the anti-microbial compounds or even the microbes themselves.

In use, for example in a hospital, health care setting, home care setting or for home or many other contexts of use, each layer is removed in sequence, exposing a new layer underneath. The layers are provided with tabs which are not adhered to one another, or which even only partially overlap: the tabs are pulled and the peelable layer is pulled off with the tab. After a suitable period of usage, the new layer is in turn removed to expose yet another later. When the final layer is removed the disposable tray is removed from the bedside/couch/chair/framed platform table and replaced with a fresh tray.

In embodiments, the core bedside table unit may sit on the floor, mount to a wall rail, or may have a projection dimensioned and configured to pass between the bottom of a mattress and the top of a box spring.

The backing layer may be formed by vacuum molding, or by other methods. The disposable layers may be sprayed or screen printed onto the backing layer, or may be manufactured separately and adhered thereto.

It is therefore an aspect, advantage, objective and embodiment of the present invention, in addition to those described above, to provide a disposable sanitary tray comprising:

a backing, the backing being made of a resilient material able to bear weight, the backing formed into a tray shape having an edge and having a top surface;

a plurality of hygienic layers, each hygienic layer comprising a film and a coating, each coating being one member selected from the group consisting of: anti-microbial coatings, anti-bacterial coatings, bacterial detection coatings, adhesives, and combinations thereof.

It is therefore an aspect, advantage, objective and embodiment of the present invention, in addition to those described above, to provide a disposable sanitary tray further comprising: at least one depression, the depression defined by the backing and conformed to by the plurality of hygienic layers, the at least one depression dimensioned and configured to accept one edge of a personal electronic device.

It is therefore one aspect, advantage, objective and embodiment of the present invention, in addition to those described above, to provide a disposable sanitary tray further comprising: at least one depression, the depression having an opening defined by a void of the backing, with a bag-like pocket passing through the voice of the backing, the bag-like pocket defined by the plurality of hygienic layers.

It is therefore one aspect, advantage, objective and embodiment of the present invention, in addition to those described above, to provide a disposable sanitary tray wherein the backing and the hygienic layers are the same, the hygienic layers offering the tray structural support.

In addition to which it is an aspect, advantage, objective and embodiment of the present invention, in addition to those described above, to provide a disposable sanitary tray for use with a liquid such as spilled beverages, bodily fluids or the like, wherein the tray has a lowest point, the lowest point being within one or more of the bag-like pockets,

the tray being dimensioned to allow such liquid to flow across at least a portion of the tray to the lowest point within one or more bag-like pockets,

whereby such liquid on the surface of the tray may be disposed of by peeling a single hygienic layer from the tray edge first, so that such liquid remains contained within one or more bag-like pockets while the single hygienic layer is removed from the tray.

It is therefore another aspect, advantage, objective and embodiment of the present invention, in addition to those described above, to provide a disposable sanitary tray for use with a liquid such as spilled beverages, bodily fluids or the like, wherein:

at least a first one of the hygienic layers is perforated so as to allow such liquid to pass through, and at least a second one of the hygienic layers is directly underneath the first hygienic layer and is comprised of an absorbent material.

It is therefore one aspect, advantage, objective and embodiment of the present invention, in addition to those described above, to provide a disposable sanitary tray further comprising:

a first capillary layer perforated by capillary passages;

a second antimicrobial layer impregnated with an antimicrobial liquid, whereby the antimicrobial liquid may pass upward through the first capillary layer to the surface.

It is therefore yet one more aspect, advantage, objective and embodiment of the present invention, in addition to those described above, to provide a disposable sanitary tray, wherein the film has antimicrobial properties and the coating is omitted.

It is therefore one more aspect, advantage, objective and embodiment of the present invention, in addition to those described above, to provide a disposable sanitary tray wherein the film comprises a material which is one member selected from the group consisting of: polymer films, metallized polymer films, metal foils, paper, cloth and combinations thereof.

It is therefore one more aspect, advantage, objective and embodiment of the present invention, in addition to those described above, to provide a disposable sanitary tray further comprising:

a second coating selected from the group consisting of: anti-microbial coatings, anti-bacterial coatings, microbial detection coatings, bacterial detection coatings, adhesives, and combinations thereof.

And it is therefore one aspect, advantage, objective and embodiment of the present invention, in addition to those described above, to provide a disposable sanitary tray wherein the anti-bacterial coating comprises one member selected from the group consisting of: antibiotic compounds, essential oils, copper, metals, disinfectants, sanitizers, bactericidal agents, bacteriostatic agents, antifungal agents, antiviral agents, antiparasitic agents and combinations thereof.

And it is therefore one aspect, advantage, objective and embodiment of the present invention, in addition to those described above, to provide a disposable sanitary tray further comprising:

a reservoir having therein a liquid antimicrobial coating.

And it is therefore one aspect, advantage, objective and embodiment of the present invention, in addition to those described above, to provide a disposable sanitary tray wherein the liquid antimicrobial coating further has current passing therethrough, light passing therethrough, UV light passing therethrough, beneficial microorganisms and combinations thereof.

And in addition to that it is therefore one aspect, advantage, objective and embodiment of the present invention, in addition to those described above, to provide a disposable sanitary tray wherein the antimicrobial coating covers a first portion of the tray top surface, the first portion being one member selected from the group consisting of: a spider web of thin lines, less than half of the top surface, more than half of the top surface, all of the top surface.

It is therefore a second or higher numbered aspect, advantage, objective and embodiment of the present invention, in addition to those described above, to provide a bedside table, TV tray, night stand, coffee table or end table comprising:

a core unit having at least one arm segment, the at least one arm segment dimensioned and configured to project over such bedside;

a connector unit mounted upon the core unit at a first end of the at least one arm segment;

a backing, the backing being made of a resilient material able to bear weight, the backing formed into a tray shape, the tray shape dimensioned and configured to mechanically engage such connector unit;

a plurality of hygienic layers, each hygienic layer comprising a film and a coating, each coating being one member selected from the group consisting of: anti-bacterial coatings, bacterial detection coatings, adhesives, and combinations thereof.

It is therefore another aspect, advantage, objective and embodiment of the present invention, in addition to those described above, to provide a bedside table, TV tray, night stand, coffee table or end table comprising:

an electronic connector suitable for connecting an electronic device to.

It is therefore yet another aspect, advantage, objective and embodiment of the present invention, in addition to those described above, to provide a bedside table, TV tray, night stand, coffee table or end table wherein the connector unit is a pair of tines and the electronic connector unit is one member selected from the group consisting of: an electrical power cord, an electronic data cable, electrical controls, low voltage connectors suitable for passing low voltage through the films, UV lighting, ambient lighting, and combinations thereof.

It is therefore another aspect, advantage, objective and embodiment of the present invention, in addition to those described above, to provide a bedside table, TV tray, night stand, coffee table or end table further comprising: low voltage conductivity through the compounds of/in the film.

It is therefore another aspect, advantage, objective and embodiment of the present invention, in addition to those described above, to provide a bedside table, TV tray, night stand, coffee table or end table wherein the hygienic layers are transparent in UV and wherein the table further comprises: a UV light disposed so as to shine through the tray.

It is therefore another aspect, advantage, objective and embodiment of the present invention, in addition to those described above, to provide a bedside table, TV tray, night stand, coffee table or end table for use with a floor or a mattress and further comprising:

a foot, the foot having a shape selected from the group consisting of: a foot dimensioned and configured to support the table when the foot is flat on such floor, a foot dimensioned and configured to support the table when the foot is inserted beneath such mattress and combinations thereof.

### BRIEF DESCRIPTION OF THE DRAWINGS

FIG. 1 is a side view of a disposable sanitary tray of the invention showing a first preferred embodiment.

FIG. 2 is a top planform view of the disposable sanitary tray of the invention's first preferred embodiment, showing additional features thereof.

FIG. 3 is a side view of the disposable sanitary tray of the invention showing a first preferred embodiment with the tabs of the peelable layers shown.

FIG. 4 is a top planform view of the disposable sanitary tray of the invention showing a first preferred embodiment with the tabs of the peelable layers shown.

FIG. 5 is an end view of the upper portion of a bedside table second embodiment of the invention having a somewhat simpler disposable tray thereon.

FIG. 6 is a side elevational perspective view of the second embodiment bedside table and disposable tray, showing the tabs and layers thereof.

FIG. 7 is a side elevational perspective view of the disposable tray in a third embodiment.

FIG. 8 is a corner cutaway showing multiple layers of film.

FIG. 9 is a side (partially cutaway) view of a core bedside table element of a fourth embodiment of the invention, showing the arm of the bedside table extended and with no base shown, for clarity.

FIG. 10 is a side view, partially cutaway, of the bedside table of a fifth embodiment of the invention, showing the floor base unit for support.

FIG. 11 is a side, partially cutaway view of the bedside table according to a sixth embodiment of the invention, showing the wall mounts such as might be used in an institutional setting.

FIG. 12 is a side and partially cutaway view of the bedside table according to a seventh embodiment of the invention, showing the mattress interstitial insertion support of the invention, such as might be used in a home setting or in a home care setting.

FIG. 13 is a top planform view of a layer of disposable sanitary tray, showing how a pattern of different anti-bacterial compound and bacterial detection compound and adhesives might be laid down in a very simplified way according to an eighth embodiment of the invention.

FIG. 14 is a top planform view of a layer of disposable sanitary tray in a ninth embodiment, showing how stripes of a pattern of different types of compounds might be laid down, and further showing that the pattern may relate to parts of the tray, for example, a bacteria detection stripe might be specifically associated with a cup holder.

FIG. 15 is a top planform view of another alternative embodiment of the forks of the invention.

FIG. 16 is an end view of the forks of this alternative embodiment of the forks of the invention.

FIG. 17 is an end view of the forks of this alternative embodiment of the forks of the invention.

FIG. 18 is a top planform view of another alternative embodiment of the forks of the invention showing data/power points.

FIG. 19 is an end view of the forks of this alternative embodiment of the forks of the invention.

FIG. 20 is an end view of the forks of this alternative embodiment of the forks of the invention.

FIG. 21 is a top planform view of another alternative embodiment of the forks of the invention showing that such power connections may also serve lights placed on the invention.

FIG. 22 is an end view of the forks of this alternative embodiment of the forks of the invention. It will be noted that with a transparent or translucent tray placed on the prongs, a pretty lighting effect may be achieved.

FIG. 23 is an end view of the forks of this alternative embodiment of the forks of the invention, with the lights clearly visible.

FIG. 24 is a top planform view of another alternative embodiment of the forks of the invention showing that such power connections may also serve circular sensors on the invention.

FIG. 25 is an end view of the forks of this alternative embodiment of the forks of the invention.

FIG. 26 is an end view of the forks of this alternative embodiment of the forks of the invention.

FIG. 27 is a top planform view of another alternative embodiment of the forks of the invention showing that such power and rail connections may support even fairly large devices, such as an audio, video or laser output device.

FIG. 28 is an end view of the forks of this alternative embodiment of the forks of the invention, with the output device attached.

FIG. 29 is an end view of the forks of this alternative embodiment of the forks of the invention.

FIG. 30 is a top planform view of another alternative embodiment of the forks of the invention showing a net embodiment.

FIG. 31 is an end view of the forks of this alternative embodiment of the forks of the invention, with the net attached and showing further details of how accessories may attach to the rails.

FIG. 32 is an end view of the forks of this alternative embodiment of the forks of the invention.

FIG. 33 is a front perspective elevational view of another alternative tray embodiment of the invention.

FIG. 34 is a top planform view of this alternative tray embodiment of the invention.

FIGS. 35-38 are a repeat of FIGS. 21, 22, and 23.

FIG. 38 is an elevational side perspective view of another alternative embodiment of the locking mechanism of the fork of the invention.

FIG. 39 is a top view in partial transparency of the same embodiment of the fork lock mechanism alternative.

FIG. 40 is a partially transparent side view of the alternative embodiment of the fork locking mechanism, showing details of how the lock works, as well as adding details of controls (buttons), a data outlet and so on.

FIG. 41 is a side view showing how one arm embodiment of the invention may fold conveniently out of the way, or may be made as short or long as necessary.

FIG. 42 is atop view of an arm segment of one embodiment of the invention.

FIG. 43 is a side view of an arm segment of the same embodiment of the invention as shown in FIG. 42.

FIG. 44 is an end view of an arm or prong segment, demonstrating how power/data cables may be easily disposed therein. Thus a rubber plug as shown above, or rubber flanges as shown below, may be employed to retain cabling, and yet also allow easy access, removal, addition, etc.

FIG. 45 is a view showing further details of the arm arrangements of the invention, including how cabling may be arranged, specifically spiraled cord suitable for being stretched and retracted with an arm.

FIG. 45 is a view showing further details of the arm arrangements of the invention, including how cabling may be arranged, specifically spiraled cord suitable for being stretched and retracted with an arm.

FIG. 46 is a view showing further details of the arm arrangements of the invention, including how cabling may be arranged, specifically spiraled cord suitable for being stretched and retracted with an arm.

FIG. 47 is a view showing further details of the arm arrangements of the invention, including how cabling may be arranged, specifically spiraled cord suitable for being stretched and retracted with an arm.

FIG. 48 is a view showing further details of the arm arrangements of the invention, including how cabling may be arranged, specifically spiraled cord suitable for being stretched and retracted with an arm.

FIG. 49 is a view showing further details of the arm arrangements of the invention, including how cabling may be arranged, specifically spiraled cord suitable for being stretched and retracted with an arm.

FIG. 50 is a view of the upper rail embodiment of the invention.

FIG. 51 is a side view of the upper rail embodiment of the invention.

FIG. 52 is a view of the lower rail embodiment of the invention.

FIG. 53 is a side view of the lower rail embodiment of the invention.

FIGS. 54a and 54b are top and side views similar to FIG. 14 but with a different arrangement of coatings on a layer.

FIG. 55 is a side view showing that the swing arm can fold in on the cylinder.

FIG. 56 is a side view showing that the swing arm can fold in on the cylinder.

FIG. 57 is atop view of an embodiment having additional friction control devices (raised areas) thereon.

FIG. 58 is a side view of the mattress insert support.

FIG. 59 is a side view of the mattress insert support.

FIG. 60 is a side view in partial cutaway of a strut of the invention.

FIG. 61 is a side view in partial cutaway of a strut of the invention.

FIG. 62 is a top view of the claw stand of the invention in another embodiment.

FIG. 63 is a side view of the claw stand of the invention in another embodiment.

FIG. 64 is a rear view of the claw stand of the invention in another embodiment.

FIG. 65 is atop view of a tray of the invention having perforations in the topmost visible layer.

FIG. 66 is a side view of a tray of the invention having bags passing through some of the holes.

FIG. 67 is a side view of a tray of the invention half way through the process of having a film removed so as to form a bag for liquids at the lowest point in the tray.

FIG. 68 is a side cross-sectional view of one exemplary arrangement of layers of the invention having two or more coatings per layer.

FIG. 69 is a side cross-sectional view of one exemplary arrangement of layers of the invention having perforations with an absorbing layer beneath.

FIG. 70 is a side cross-sectional view of one exemplary arrangement of layers of the invention having perforations with a liquid antimicrobial compound able to wick, flow or flow by capillary action upward through the perforated layer above.

FIG. 71 is a top view of a tray having the coating applied in a spider web pattern.

FIG. 72 is a side view of an embodiment in which the tray of the device is also a liquid storage reservoir or tank for the antimicrobial compounds.

FIG. 73 is a top view of an embodiment in which the tray of the device is also a liquid storage reservoir for the antimicrobial compounds.

INDEX OF REFERENCE NUMERALS

| | |
|---|---|
| Disposable tray | 100 |
| Feature | 102 |
| Cup holder indentation | 104 |
| Tablet holder feature | 106 |
| Stepped tablet holder | 108 |
| Anti-bacteria film | 200 |
| Tab | 202 |
| Tab | 204 |
| Arrow | 206 |
| Backside/back layer of film/adhesive layer | 208 |
| Arrow | 210 |
| Backside/back layer of film/adhesive layer | 212 |
| Disposable Tray | 300 |
| Detail of Tray/Table | 302 |
| Tabs | 304 |
| Backside/back layer of film/adhesive layer | 306 |
| Hospital Bedside Table | 308 |
| Main unit | 400 |
| Tines (lined up) | 402 |
| Latch | 404 |
| Pivot/lock | 406 |
| Arm segment | 408 |
| Recess | 410 |
| Cord | 412 |
| Arm segment | 414 |
| Piston Segment | 416 |
| Floor base | 520 |
| Forks (side on) | 522 |
| Tray | 524 |
| Connector (power cord) | 526 |
| Wall/floor | 630 |
| Wall rail/track | 632 |
| Connector | 634 |
| Mattress insert support | 702 |
| Anti-bacterial compound | 802 |
| Bacterial detection compound | 804 |
| Adhesive | 806 |
| Stripe of anti-bacterial compound/adhesive | 902 |
| Stripe of bacterial detection compound/adhesive | 904 |
| Film | 1004 |
| Pore | 1006 |
| Tray | 1102 |
| Bag-like pocket | 1104 |
| Tray | 1202 |
| Bag | 1204 |
| Removed parts | 1206 |
| Liquid | 1208 |
| Solid film | 1302 |
| First coat | 1304 |
| Second coat | 1306 |
| Solid film | 1402 |
| Spongy layer | 1404 |
| Perforated layer | 1406 |
| Pore | 1408 |
| Solid layer | 1502 |
| Liquid antimicrobial | 1504 |
| Perforated layer | 1506 |
| Pore | 1508 |
| Spider web pattern coating | 1604 |
| Tray | 1702 |
| Tank/shell | 1704 |
| Tank/shell | 1706 |

### DETAILED DESCRIPTION

It is therefore a preferred embodiment and best mode now contemplated for carrying out the invention to provide a disposable tray as part of a bedside table, the disposable tray having numerous layers of removable material thereon so that as one layer is used, or dirtied, or simply due to the passage of a period of time, a layer may be removed—along with any micro-organisms clinging thereto—and a new layer is exposed, clean. The layers may additionally have anti-microbial compounds on them, such as anti-fungal or anti-bacterial compounds. Detection compounds may be used, liquids, lights, and time release compounds may be used.

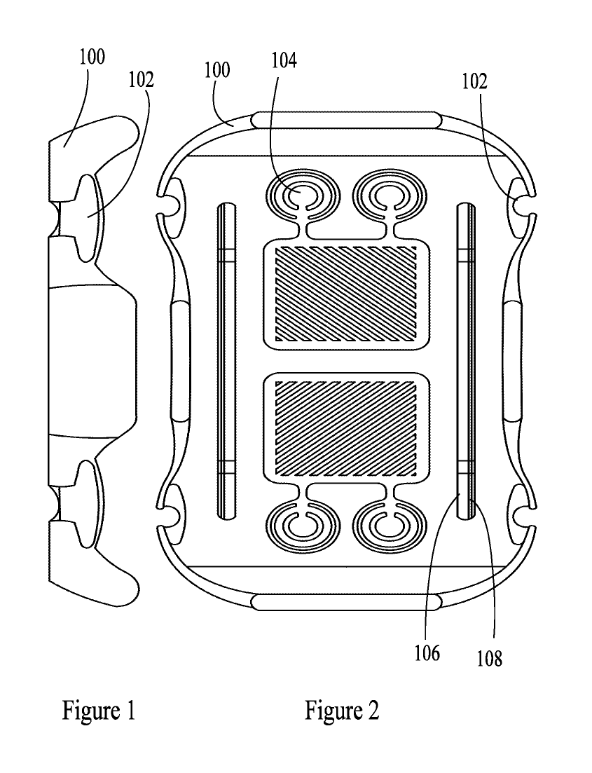

FIG. 1 is a side view of a disposable sanitary tray of the invention showing a first preferred embodiment, while FIG. 2 is a top planform view of the disposable sanitary tray of the invention's first preferred embodiment, showing additional features thereof. Disposable tray 100 is dimensioned and configured for convenient use with or as a bedside table. For example, it may have features such as feature 102, or cup holder indentation 104 or tablet holder feature 106, whose uses are generally clear: cup holder indentation 104 may hold the base of a cup so that if the bedside table is jostled, the cup is not spilled. Tablet holder 106 allows one edge of a tablet device to be inserted and then held in place.

Stepped tablet holder 108 has a bottom contour which is dimensioned and configured to accept different thicknesses and shapes (and thus brands) of tablets. The bottom contour may have a larger section into which a first tablet's edge may sit, and then may have additional steps which will catch and hold the edges of other tablets having differing thicknesses or sections.

The shape of the bottom contour and cupholders of the embodiment may be dimensioned and configured to allow liquids to flow into the center (for example, the two large rectangular areas). When the device is peeled away, the user (such as a nurse, etc) may peel the edges first and the center last, thus allowing the peel away coating of plastic, metal, copper, foil, etc, function as a bag and the spilled liquids may be bagged and taken away without any need to wipe down the tray.

Films may have texturing or microchannels to enhance the flow of the anti-microbial compounds or even the microbes themselves.

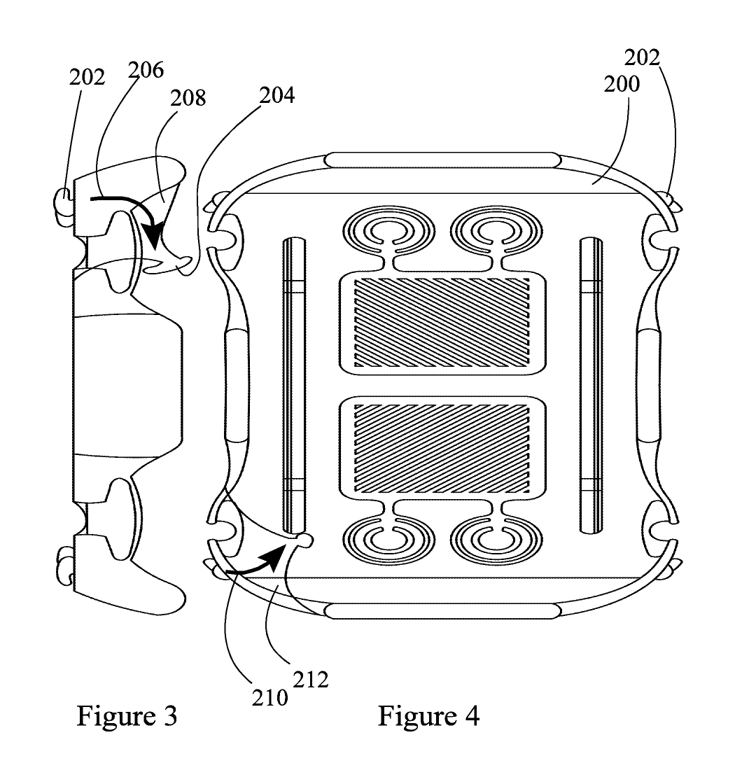

FIG. 3 is a side view of the disposable sanitary tray of the invention showing a first preferred embodiment with the tabs of the peelable layers shown, while FIG. 4 is a top planform view of the disposable sanitary tray of the invention showing a first preferred embodiment with the tabs of the peelable layers shown.

Anti-bacteria film 200 will cover a substantial part of the surface of the disposable tray, for example, in the preferred embodiments it may cover the entire top surface of the tray.

Tab 202 and tab 204 may have no adhesive thereon. The tabs 202/204 may conveniently project from a feature or from an edge of the tray so that they can easily be individually gripped between thumb and forefinger. When the tab which is part of the topmost layer, or is connected to the topmost layer, is gripped and pulled as shown by arrow 206/arrow 210, the pull will lift the immediately adjoining areas of the layer up and off the disposable tray. Continuing to pull, larger and larger parts of the layers will be removed until finally the entire layer can be removed. It will be appreciated that it may be advantageous, in embodiments, for the adhesive to be tacky, however the exact adhesive properties will depend upon other aspects of the embodiment.

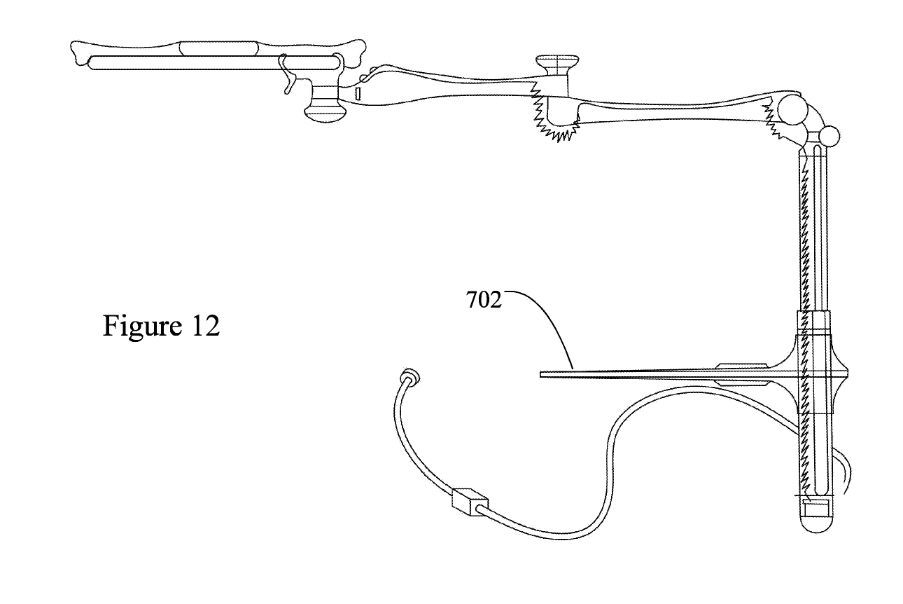

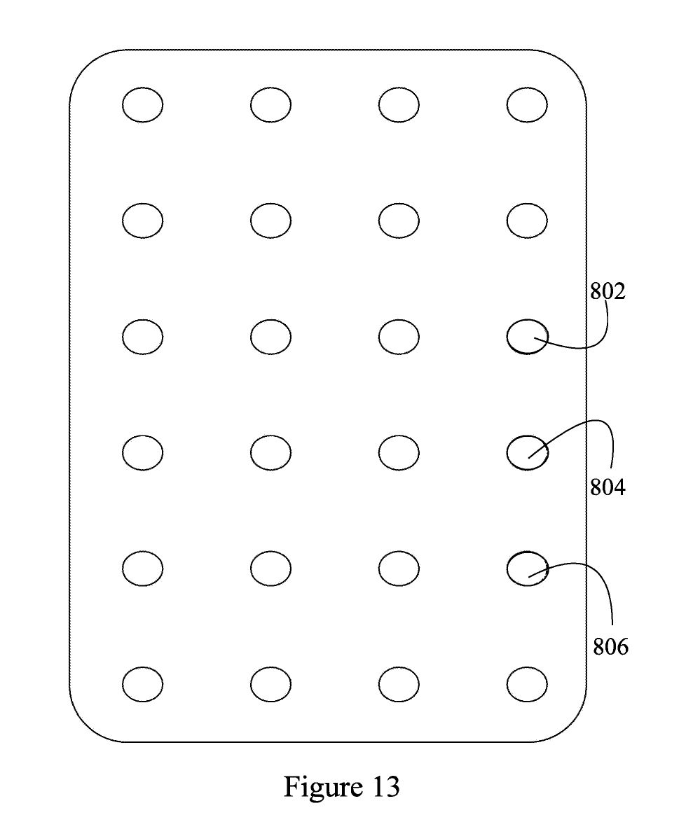

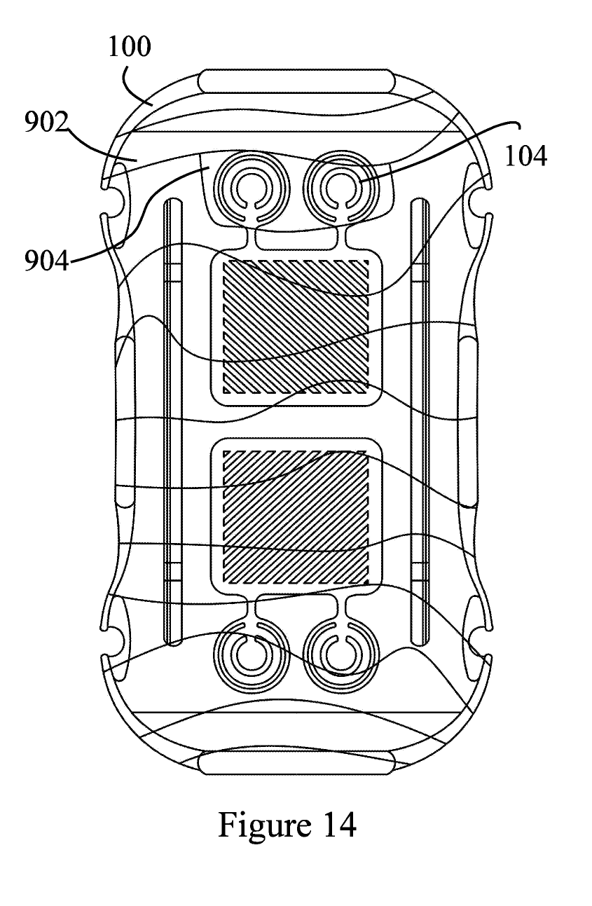

Backside/back layer of film/adhesive layer 208 and similar layer 210 may be provided with an adhesive to hold the layers together and to the disposable tray prior to being used and pulled off one by one. Note that the adhesive compound underneath a first layer might negatively interact with the anti-micro-organism compound of the next layer down. However, this can be handled in several ways. Two ways are discussed further in reference to FIGS. 13 and 14: striping/segregation. Yet another way is to select an anti-bacterial/anti-micro-organism compound which is inherently sticky, or tacky, itself. Yet another way is to select an adhesive which peels off cleanly and yet does not remove any of the layer of anti-microbial compound below it.

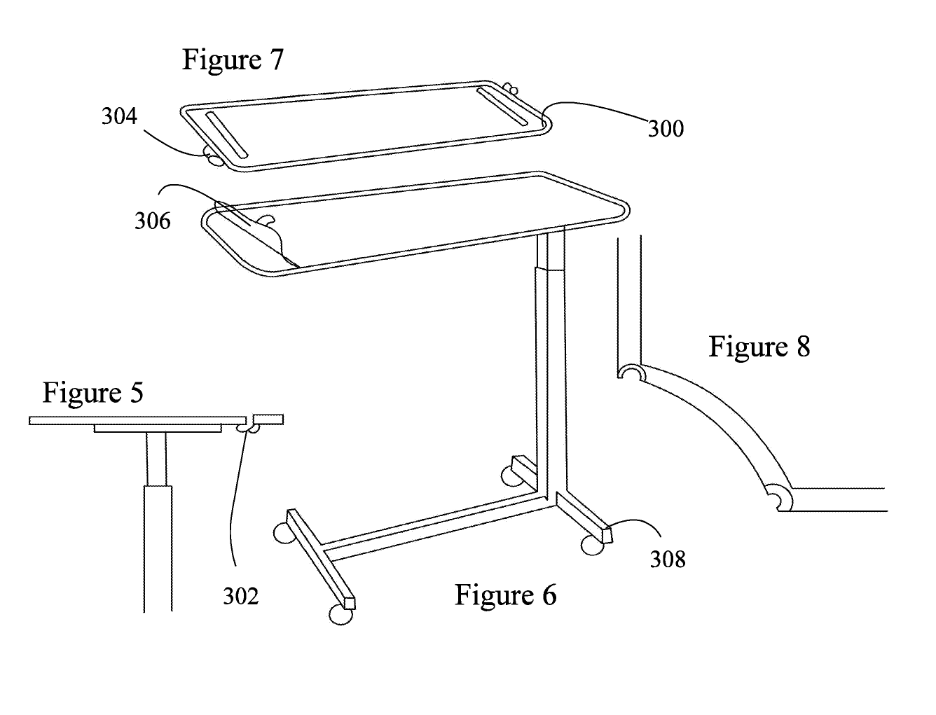

FIG. 5 is an end view of the upper portion of a bedside table second embodiment of the invention having a somewhat simpler disposable tray thereon, while FIG. 6 is a side elevational perspective view of the second embodiment bedside table and disposable tray, showing the tabs and layers thereof. In addition, FIG. 7 is a side elevational perspective view of the disposable tray in a third embodiment.

FIG. 8 is a view of the peel tabs 302 of the layers invention, a feature which makes for easy removal of a layer after a pre-determined time or when it is no longer clean.

Disposable tray 300 may be seen to be of fairly conventional shape and size for a bedside table top in this alternative embodiment. Thus the tray 300 may be used in place of metal trays or table tops which ordinarily are used beside beds such as hospital beds, hospice beds, home care beds and in similar situations.

Detail of Tray/Table 302 shows peel away tabs used to peel away layers.

Tabs 304 may be as previously described, and when pulled will expose backside/back layer of film/adhesive layer 306. In this case the pulling off of the layer may be even faster than in other embodiments due to the simpler shape of the tray.

Hospital bedside table 308 may be seen in an overall view, the table being of relatively simple shape and dimension. It has a wheeled base part which can extend underneath a bed and the disposable tray part, with attachment parts, which hangs low over the bed.

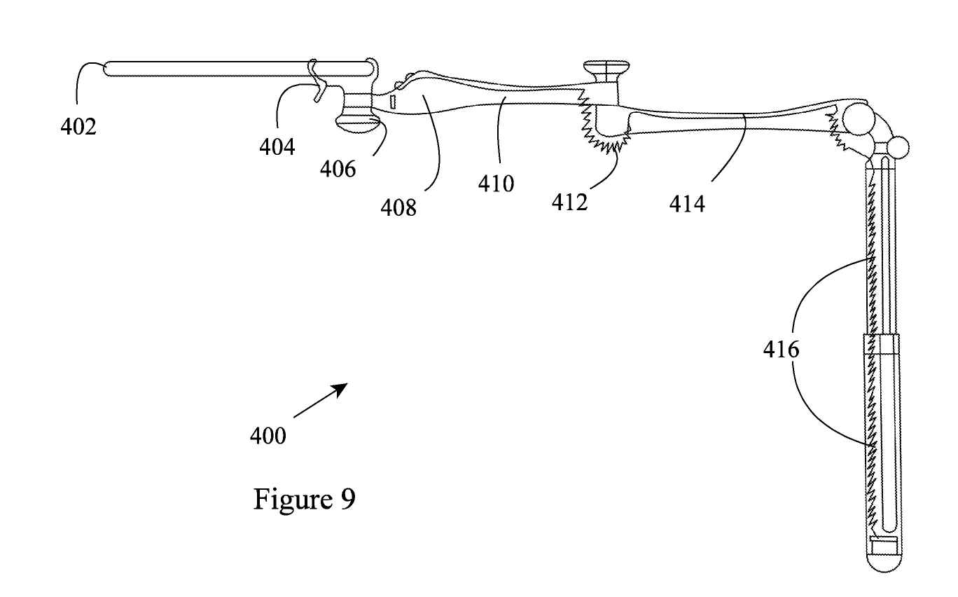

FIG. 9 is a side (partially cutaway) view of a core bedside table element of a fourth embodiment of the invention, showing the arm of the bedside table extended and with no base shown, for clarity. This device looks very different from known prior art devices. Main unit 400 can have the tray attached at one end (by means of tines 402 which appear as one because they lined up in this illustration) or by means of other attachment devices.

Latch 404 may be seen to provide a unique shape for easy fastening and unfastening, as well as being secure: this latch is adapted for the use of the disposable tray with the connector (tines in this embodiment) which hold the tray to the rest of the table.

Pivot/lock 406 allows the arm segments, such as arm segment 408 or 414, to be rotated so that the tray is held in any desired location, and yet be locked once the various parts are in that location.

Recess 410 serves several purposes, such as weight and material reduction and providing an easier grip. However, the recess 410 provides a safe secure location to tuck cord 412 so that the table can be provided with an electrical/electronic connection such as a power cord and power outlet disposed conveniently to hand on or near the disposable tray, or an electronic data cable connection such as for a USB cable or the like.

Piston Segment 416 allows the device to be raised or lowered, and in addition may assist that motion by means of mechanical linkages or a gas shock, an electric worm drive and so on and so forth. As will be seen in the next several diagrams 10 through 12, the piston segment 416 further allows a secure anchor point for the bedside table of the invention to be attached to a base or support.

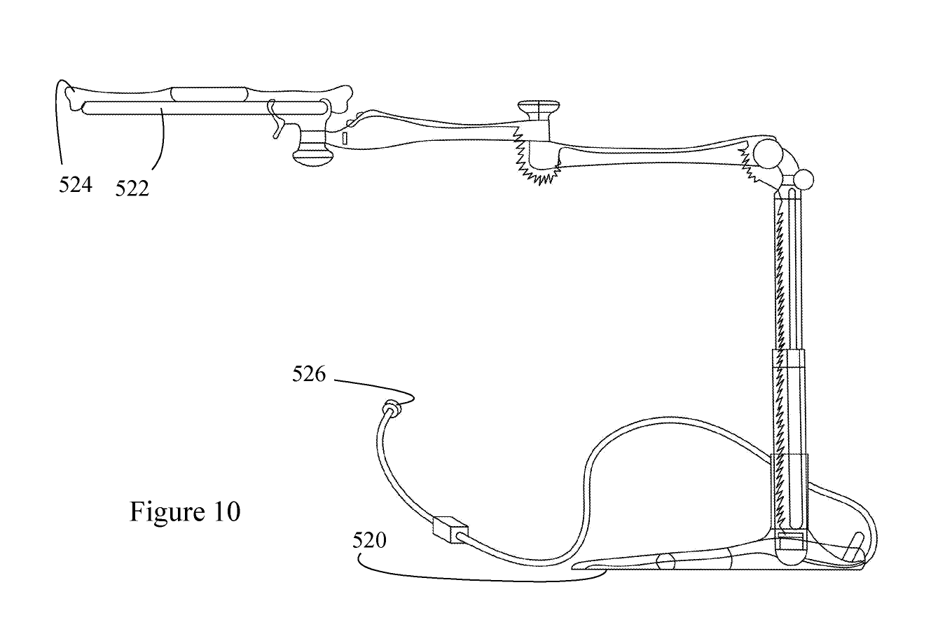

FIG. 10 is a side view, partially cutaway, of the bedside table of a fifth embodiment of the invention, showing the floor base unit for support. Floor base 520 has an elongated projection which provides leverage for stability. The floor base is also heavy, being made of solid metal or the like, so as to provide weight for additional stability.

Forks 522 (again seen side on) are dimensioned and configured, and tray 524 is likewise dimensioned and configured, so as to engage to one another mechanically, for example, the tines 522 may extend under the tray into raised areas, into springs, holes, etc in order to secure them together. The tines/connector may provide a solid feel to the tray, allowing thinner tray construction (the tray may be disposable) without sacrificing a feeling of stability.

Connector 526 (a power cord in the embodiment illustrated) may plug into a standard wall outlet.

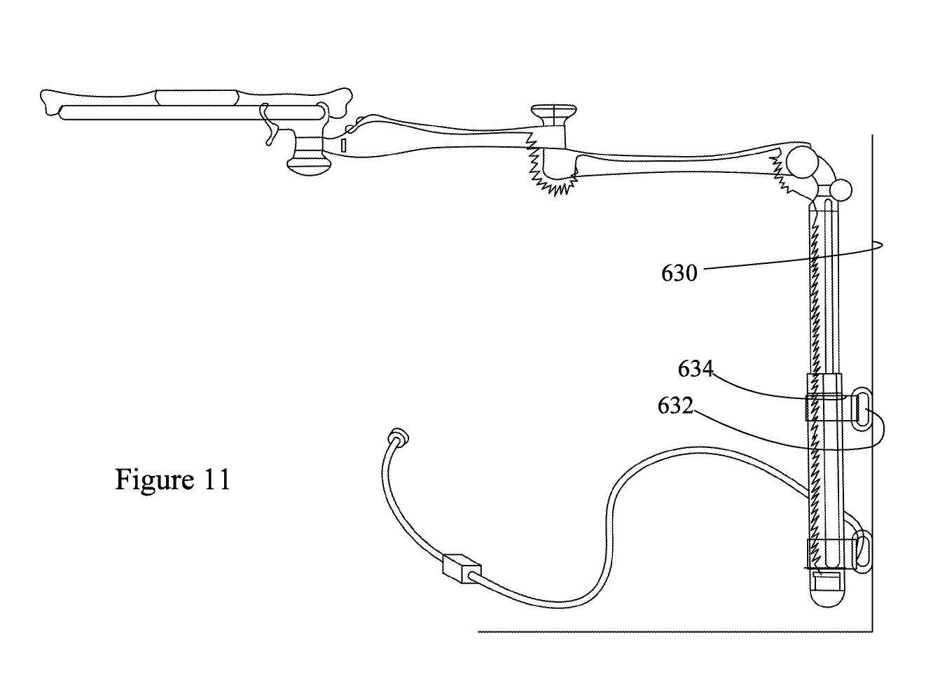

FIG. 11 is a side, partially cutaway view of the bedside table according to a sixth embodiment of the invention, showing the wall mounts such as might be used in an institutional setting.

Wall/floor 630 may be used to support wall rail/wall track 632 (or in preferred embodiments such as that depicted, more than one track) which in turn supports connector 634, which in turn is secured to the piston portion of the core unit. This embodiment may be preferable in institutional settings such as care facilities or health care facilities in which beds may be moved and rooms rearranged and yet the device should be flexibly employable in the same room in a new spot, and the device should be fastened down. It may be preferable for other reasons as well to have a hospital device mounted to a wall.

This standardized rail may also be a data rail, which allows equipment to slide along the rail freely and yet maintain a data connection between the equipment mounted on the rail and the internet or other data network.

FIG. 12 is a side and partially cutaway view of the beside table according to a seventh embodiment of the invention, showing the mattress interstitial insertion support of the invention, such as might be used in a home setting or in a home care setting.

Mattress insert support 702 is yet another unique feature of the invention. The mattress insert support 702 may be inserted interstitially (between otherwise adjacent things), in this case, the insert support 702 may slide easily in between the mattress and the box spring. It will then be held in place by the weight of the mattress above it and the friction with the mattress and box spring. It may also be inserted below the box spring, depending on the bed construction. In yet other settings, such as a single mattress care facility bed, it may be inserted between the mattress and the surface of the bed on which the mattress rests. This base has the advantages of being instantly removable and transferrable, stable, light weight and inexpensive.

While shown side on and thus appearing to be a tongue or bar, in fact in the preferred embodiment shown the insert may be a large flat device having an oval, round, rectangular or similar planform.

FIG. 13 is atop planform view of a layer of disposable sanitary tray, showing how a pattern of different anti-bacterial compound and bacterial detection compound and adhesives might be laid down in a very simplified way according to an eighth embodiment of the invention. In this alternative embodiment, the various different compounds may be separated and laid in a simple pattern. Thus the row of “dots” 802 may be anti-bacterial/anti-microbial compound, while the next row may be a bacterial detection compound 804 and the row after that adhesive 806. Obviously the areas may be shapes other than dots, the compounds used may be any suitable compounds, the area covered may be greater or less than shown and so on and so forth.

One superior example of this is the preferred embodiment of FIG. 14, which is a top planform view of a layer of disposable sanitary tray in a ninth embodiment, showing how stripes of a pattern of different types of compounds might be laid down, and further showing that the pattern may relate to parts of the tray, for example, a bacteria detection stripe might be specifically associated with a cup holder. Stripe of anti-bacterial compound/adhesive 902 may be seen near the visually upper end of the tray, while adjoining it is a stripe of bacterial detection compound/adhesive 904 which is a noticeably different shape. In fact, stripe 904 is designed to largely encircle the cup holders since this area is one which is prone to accumulating bacteria (from liquids, hands and lips) more quickly than other areas, making it an ideal location for the detection compound to be employed. Other similar stripes (or strips) may be extend across the entire surface of the tray if desired.

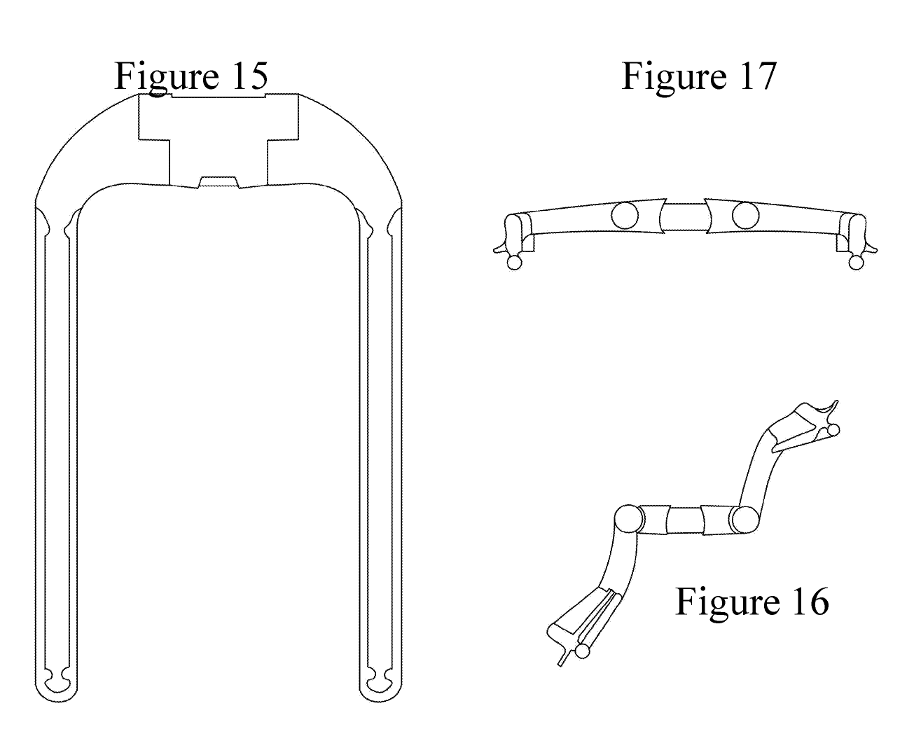

FIG. 15 is a top planform view of another alternative embodiment of the forks of the invention, while FIG. 16 is an end view of the forks of this alternative embodiment of the forks of the invention and FIG. 17 is an end view of the forks of this alternative embodiment of the forks of the invention showing how they may be hinged so as to allow them to assume different positions, which in turn allows any tray or device suspended upon them to be adjusted for the extreme comfort of the user.

As shown in FIG. 15, the recessed contour allows the fork to lock in place, while a spring loaded button lock allows the individual prongs of the forks to be moved or held stationary. Small indentations near the ends and beginnings of each fork serve as locking points for the tray, caddy, containers or provide a location for a power or data source. A spring-loaded button lock may hinge upon the prong or the fork.

FIG. 16 shows that both positive and negative angular motion is possible: the individual prongs may move independently, and FIG. 17 depicts the fact that fastening points on the forks may be employed for attachments. In this particular embodiment and in several presently preferred embodiments, these fastening points may be rails extending the length of the prongs. Accessories for the rails will be shown in later diagrams.

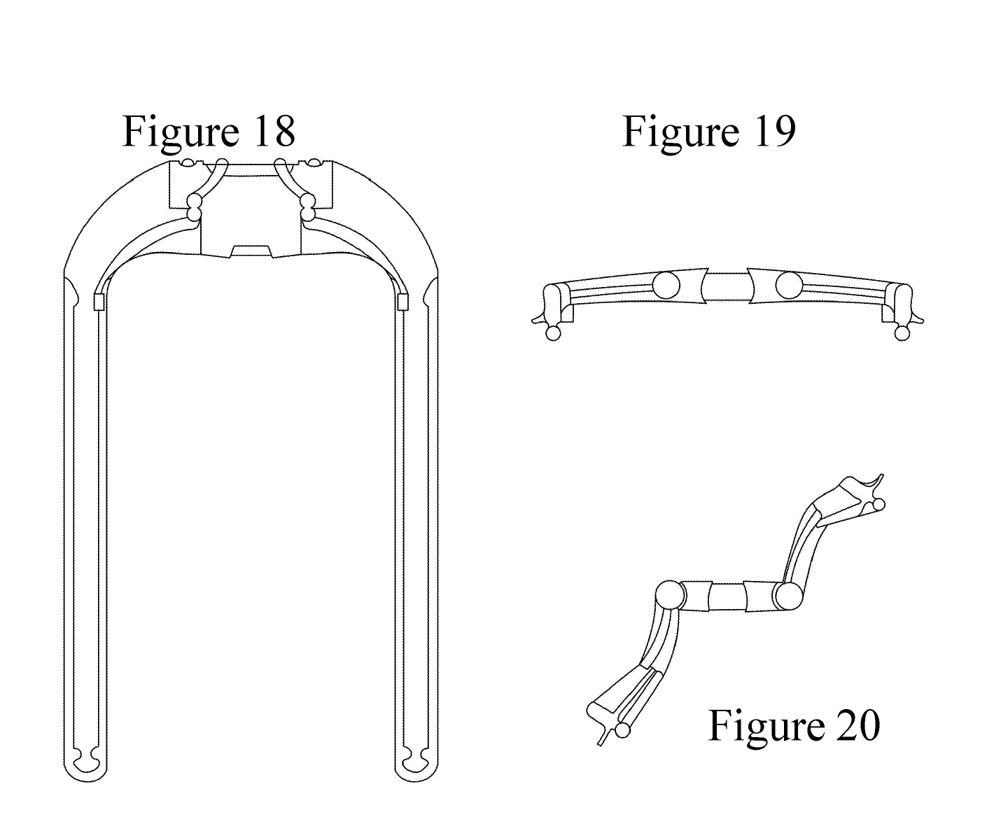

FIG. 18 is a top planform view of another alternative embodiment of the forks of the invention showing data/power points. Power and data connection points at the top of the figure have electrical/data conduits (wires) running therefrom and down into the prongs. These conduits may be below the prongs, atop them, beside them, or preferably, may be embedded within the prongs and the fork in general or placed inside of tubing. As discussed in reference to FIG. 15, the indentations may have power/data sockets therein for the convenience of the user.

FIG. 19 is an end view of the forks of this alternative embodiment of the forks of the invention while FIG. 20 is an end view of the forks of this alternative embodiment of the forks of the invention: in both cases the power outlets may be seen somewhat obscured by the prongs, but present. The power conduits may be seen clearly.

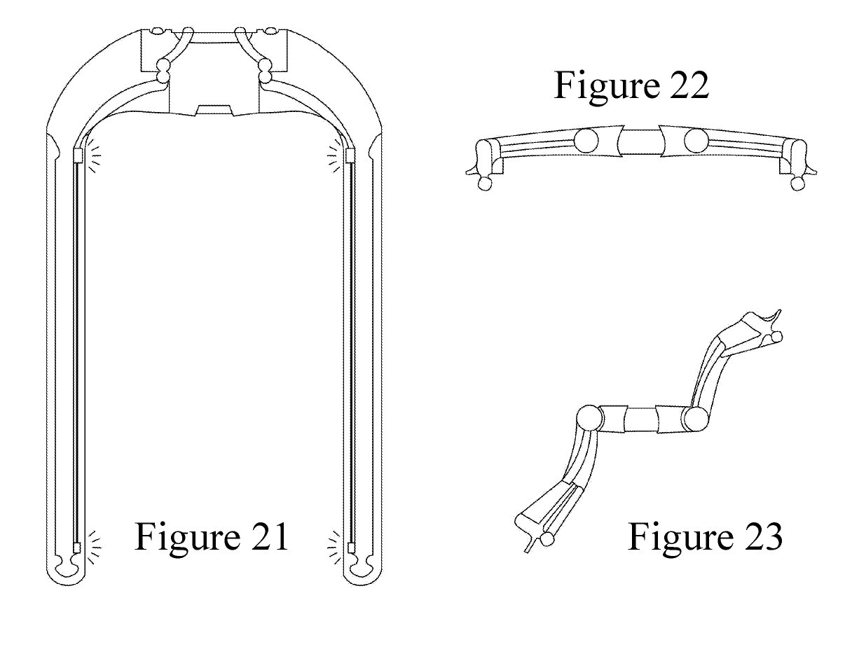

FIG. 21 is a top planform view of another alternative embodiment of the forks of the invention showing that such power connections may also serve lights placed on the invention. In particular, these lights may be placed on the inside of the prongs as shown, however, they may be anywhere on the device, including parts other than the prongs.

The lights of the invention may advantageously be UV lights designed for sanitary purposes: sterilization. The lights may also be dim or ambient lighting, or may even be reading lights (note that this particular positioning shown in FIG. 21 is not necessarily the preferred placement for reading lights, as discussed later.)

FIG. 22 is an end view of the forks of this alternative embodiment of the forks of the invention and FIG. 23 is an end view of the forks of this alternative embodiment of the forks of the invention, with the lights clearly visible. It will be appreciated that controls for electricity, data, or lighting may be part of the invention: dimmer switches, selectors, etc.

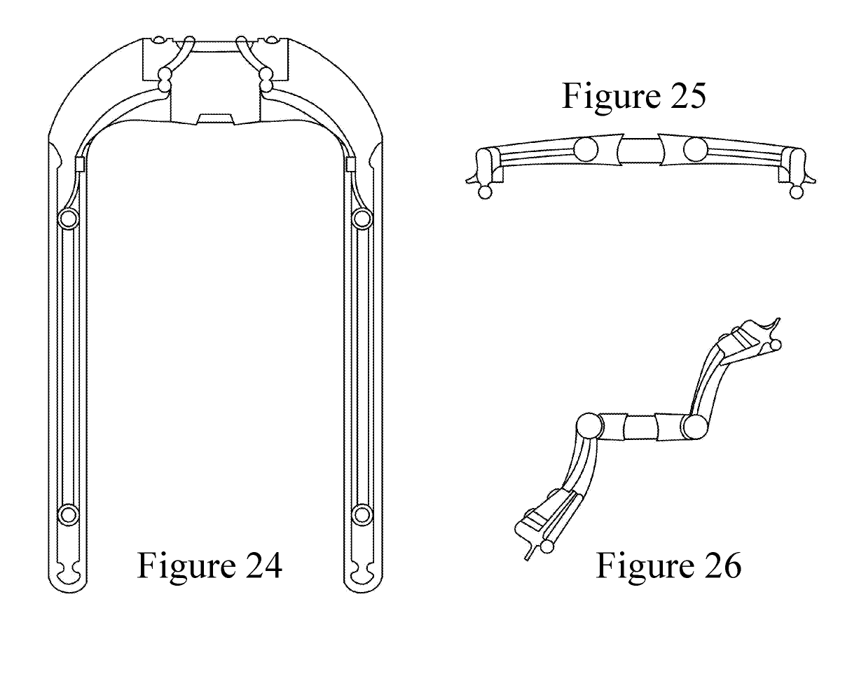

FIG. 24 is a top planform view of another alternative embodiment of the forks of the invention showing that such power connections may also serve circular sensors on the invention. While the sensors depicted are circular, located atop the prongs, and four in number there are of course no such limitations in practice. These sensors may sense pressure, weight, temperature, humidity, vapors, or may be extremely specialized sensors such as are at times used in the medical field: remote sensing pickups for bodily measurements in electrical, capacitive, particle or magnetic domains, etc. FIGS. 25 and 26 are a pair of end views of the forks of this alternative embodiment of the forks of the invention.

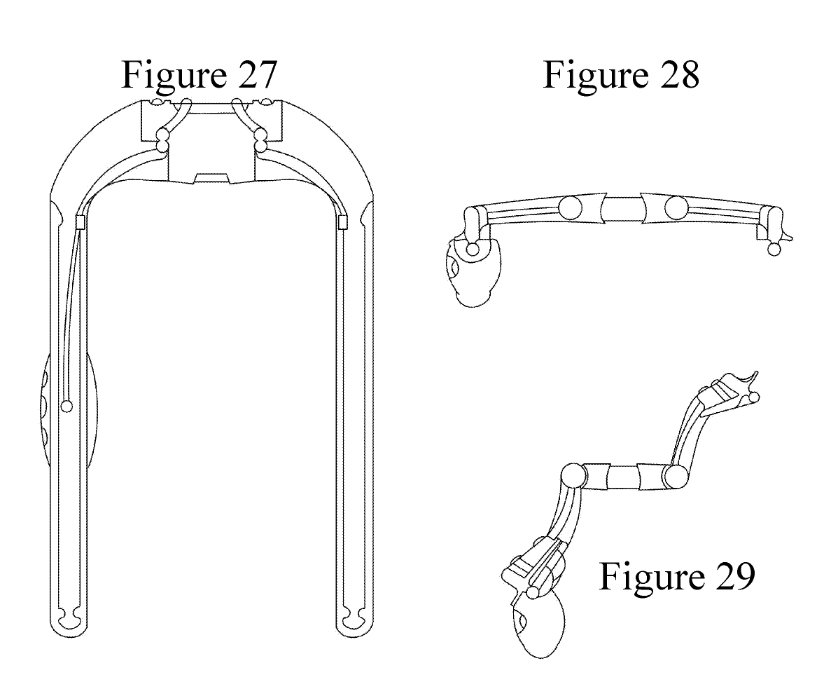

FIG. 27 is a top planform view of another alternative embodiment of the forks of the invention showing that such power and rail connections may support even fairly large devices, such as an audio, video or laser output device. The device of the invention could for example support a medical laser thermometer, or a consumer audio speaker, a small projector and so on.

FIG. 28 is an end view of the forks of this alternative embodiment of the forks of the invention, showing the output device attached. FIG. 29 is an end view of the forks of this alternative embodiment of the forks of the invention. It may be seen that the rails of the invention are used, however, any standard clamping arrangement for a device would suffice to attach it to the rails.

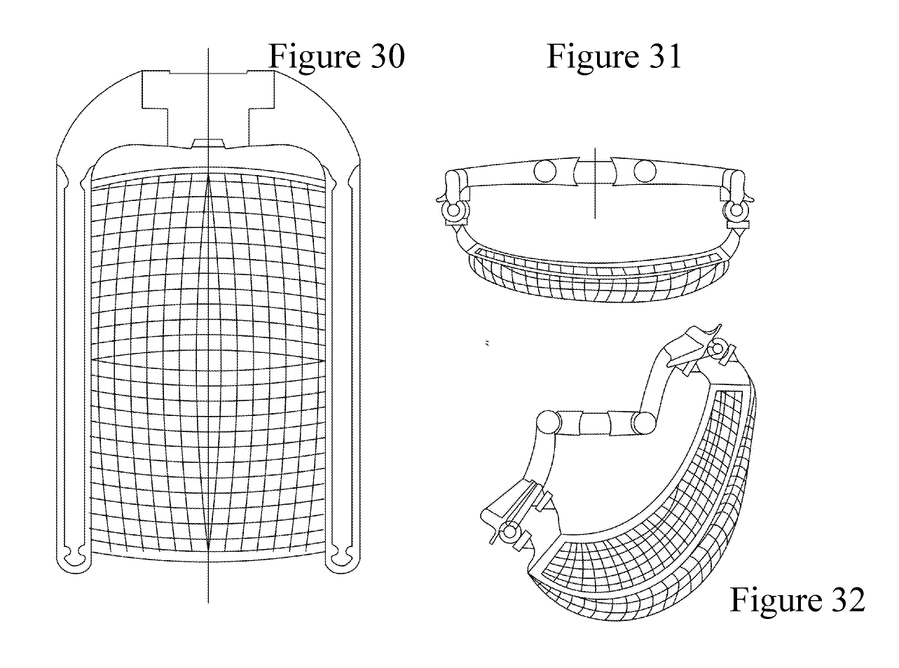

FIG. 30 is a top planform view of another alternative embodiment of the forks of the invention showing a net embodiment. This net may conveniently be used to hold articles needed by the user or by a doctor, nurse or other caregiver. It may be adjusted easily, since the prongs adjust and conform to the angle prongs.

The net may further be replaced by a shelf, of course.

FIG. 31 is an end view of the forks of this alternative embodiment of the forks of the invention, with the net attached and showing further details of how accessories may attach to the rails. A C-shaped rail may engage the rail on the prong along its length and may easily slide on and off the end as desired by the user. FIG. 32 is an end view of the forks of this alternative embodiment of the forks of the invention, also showing this. Other shapes may easily engage to the rails.

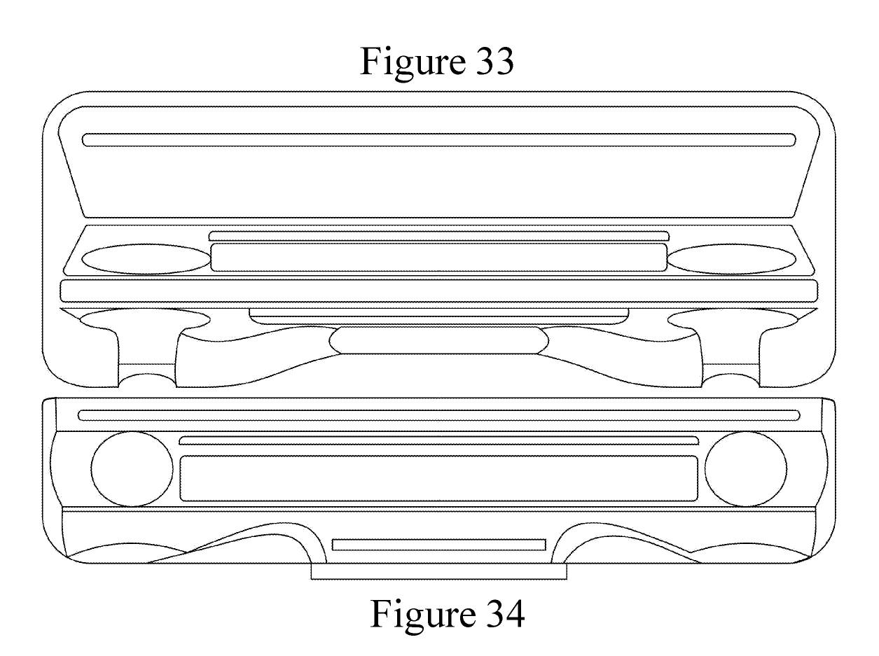

FIG. 33 is a front perspective elevational view of another alternative tray embodiment of the invention. It may be seen that the tray may advantageously be molded in highly contoured configurations, that is, while it may be similar to an airline food tray as shown in previous embodiments, it may as shown here have extensive verticals for superior support of the user's needs.

FIG. 34 is a top planform view of this alternative tray embodiment of the invention. It includes cup holders, a compartment, a tablet holder, a smart phone holder, and an indentation to slide guide the tray onto the forks and the clamp. This embodiment even has a mini-tablet holder and a handle so the entire tray may be conveniently grasped.

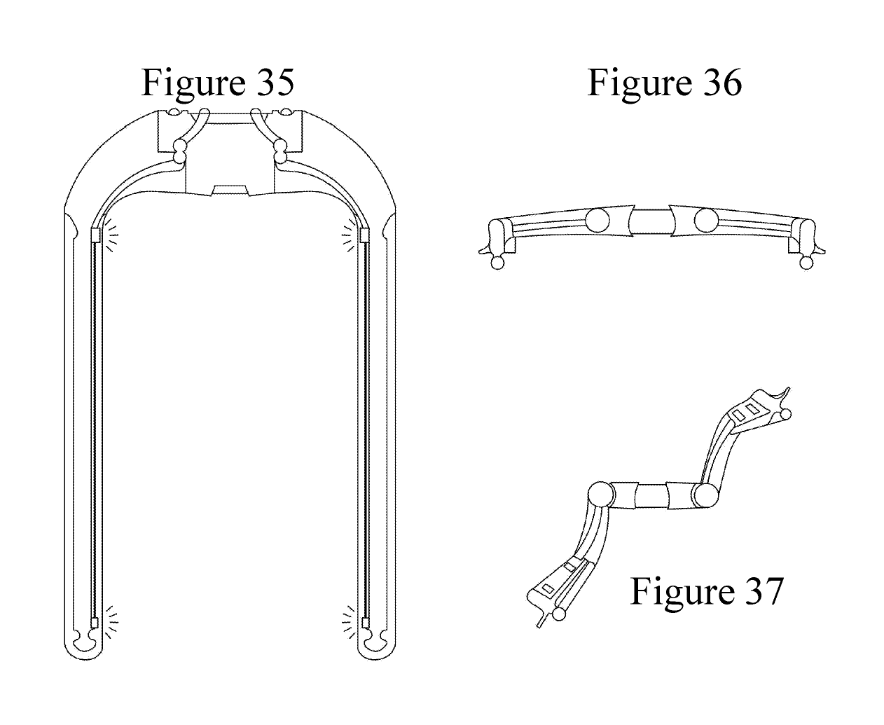

FIGS. 35, 36 and 37 are a repeat of FIGS. 21, 22 and 23.

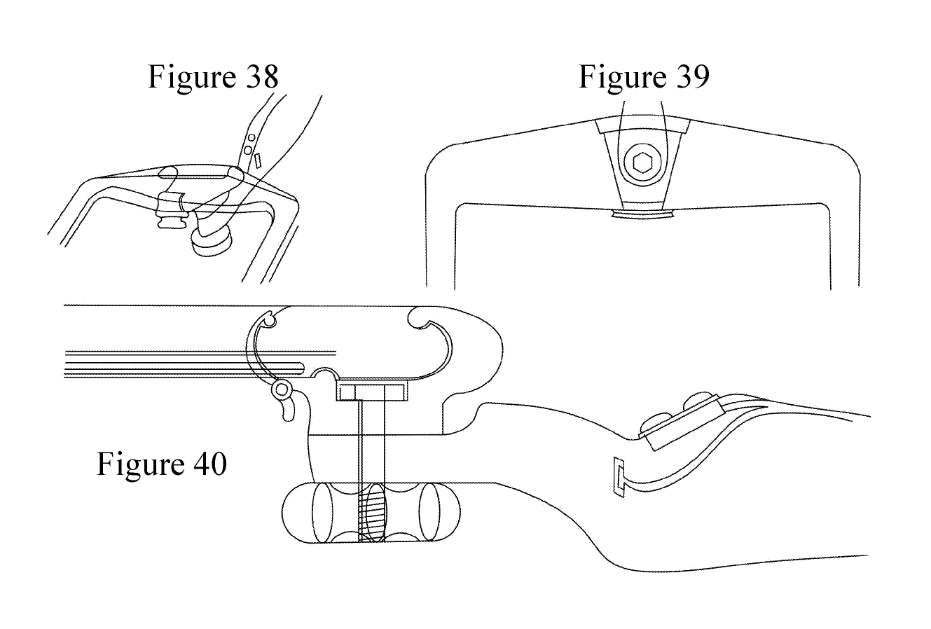

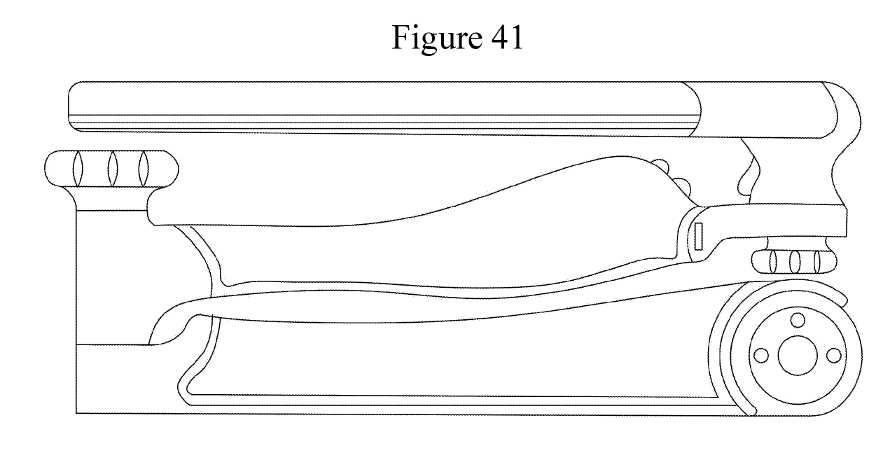

FIG. 38 is an elevational side perspective view of another alternative embodiment of the locking mechanism of the fork of the invention. The mechanism of the lock may conveniently include one or more clips as shown, hand knobs allowing a secure grip when tightening the joints of the arm and so on. In particular, the fork clamp lock (shown with the fork attached) in FIG. 38 may be lifted up to be disconnected.

FIG. 39 is atop view in partial transparency of the same embodiment of the fork lock mechanism alternative, showing the same.

FIG. 40 is a partially transparent side view of the alternative embodiment of the fork locking mechanism, showing details of how the lock works, as well as adding details of controls (buttons), a data outlet and so on. The spring loaded lock notches into the fork. Also, features of the electrical connections and swiveling are visible: the know allows a 360 degree swivel, the knob may be tightened (clicking as it does so) or loosened to allow or prevent swivel motion.

Note that another USB may be used (but not visible in this side view), and the buttons shown may even control the platform motion in the vertical dimension.

FIG. 41 is a side view showing how one arm embodiment of the invention may fold conveniently out of the way, or may be made as short or long as necessary. The three dimensional nature of the arm's joints may further be seen: by having joints which rotate in two or three dimensions it is possible for the arm to assume an extremely wide range of positions by means of the click-tension knobs at both ends which control motion in two different dimensions of rotation. The peel away tabs may actually be seen here as part of a coating of the arms, (a film, plastic, a foil, copper, etc).

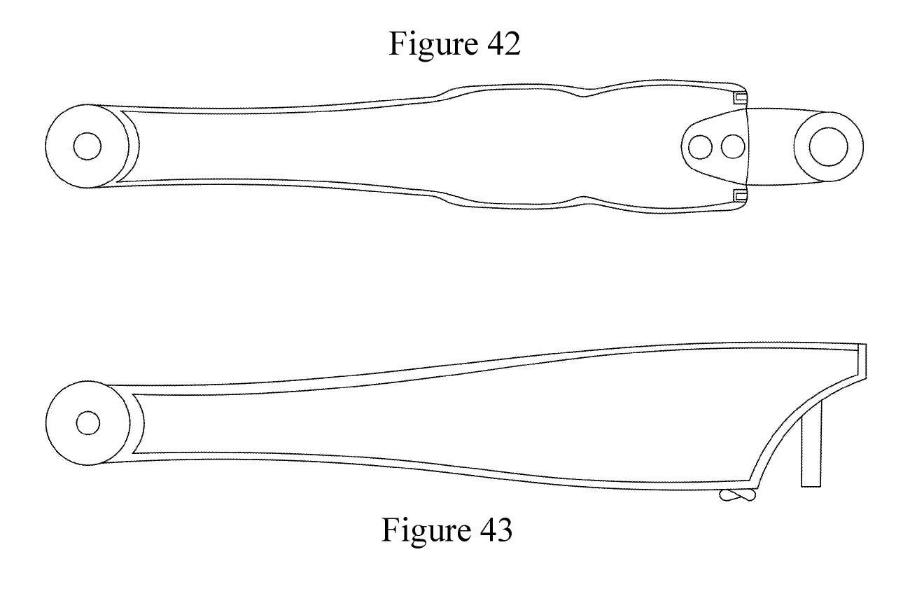

FIG. 42 is atop view of an arm segment of one embodiment of the invention, while FIG. 43 is a side view of an arm segment of the same embodiment of the invention as shown in FIG. 42. It may be seen that the arm joint may be a king pin and collar configuration. It will be seen that a film, copper and/or plastic or foil covering of the arm (rather like a skin) may further increase the easy “peel away” hygienic advantages of the device, and may have a bacterial protection compound.

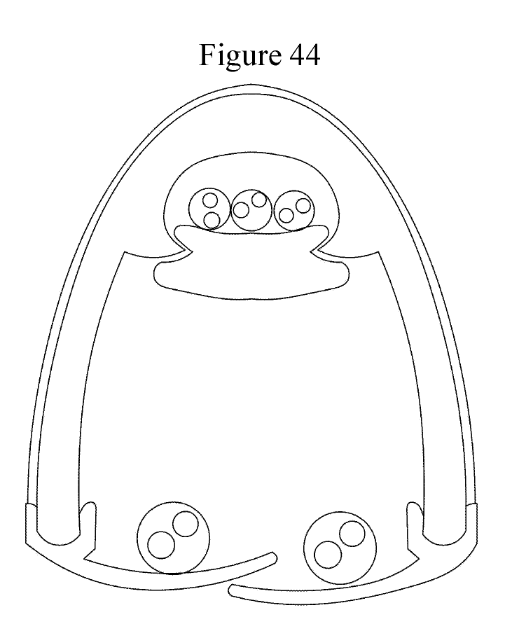

FIG. 44 is an end view of an arm or prong segment, demonstrating how power/data cables may be easily disposed therein. Thus a rubber plug as shown above, or rubber flanges as shown below, may be employed to retain cabling, and yet also allow easy access, removal, addition, etc. For example in use a person could easily reach between the flanges at the bottom, (eye-lid gaskets) which are made of a resilient flexible material, push their fingers into the space at the interior of the arm, reach upward for one of the two external cables and pull the cable free. Equally another cable can be added.

Use of the plug-style retainer or gasket is similar (this holds the upper compartment cabling), with the addition of removing the plug before altering the cords/cables. Thus there are three interior cables as well as the two exterior wires.

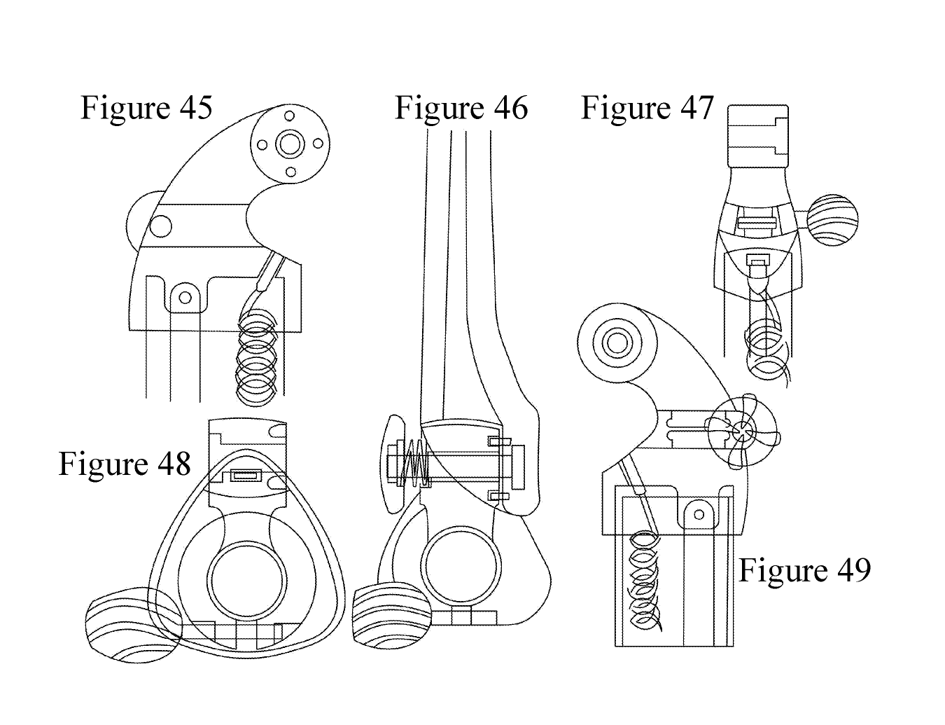

FIGS. 45-49 is a view showing further details of the arm arrangements of the invention, including how cabling may be arranged, specifically spiraled cord suitable for being stretched and retracted with an arm. Various joint views are depicted, with knurled knobs for tightening or other adjustments, the coiled cable, an outlet, and so on. FIG. 45 shows a side view of the locking mechanism with four small detents, allowing 0 to 90 degree motion. Immediately below that is the 360 degree swing point discussed earlier with reference to FIGS. 42 and 43 and the USB plug and cable. FIG. 46 shows another view of a support arm with a peel away coating, as well as showing details of the push button released support arm joint (the spring inside the push button may be seen). FIG. 47 shows a torque knob to tighten the supports arm swing, as does FIG. 49. It is worth mentioning that the shape of the cylinder can be circular in cross section or ovoid, elliptical, egg-shaped, etc.

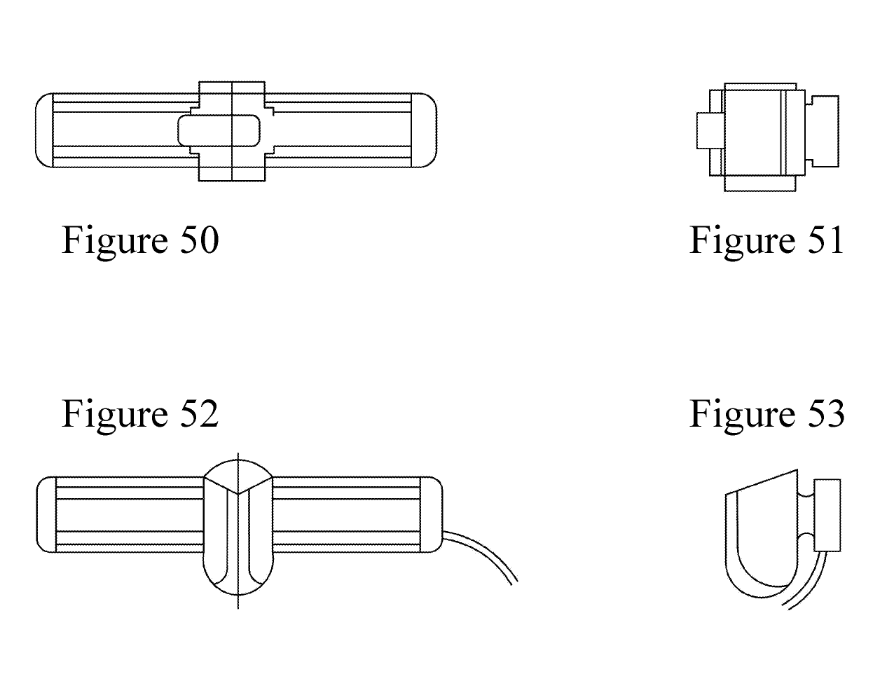

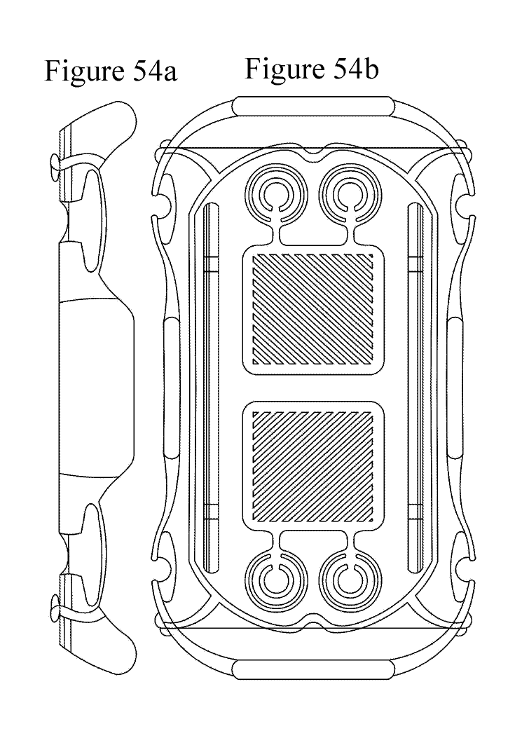

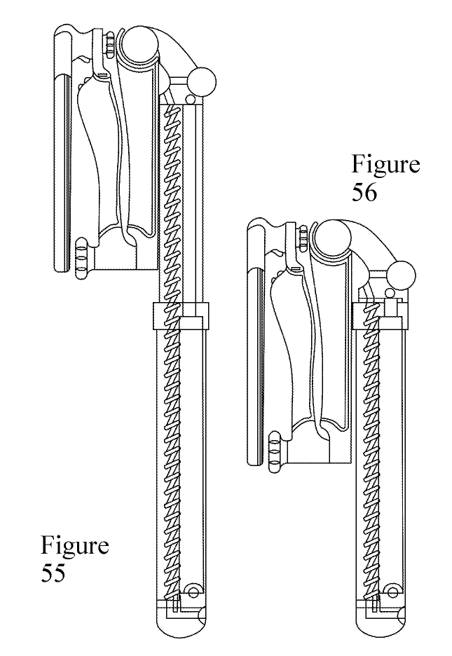

FIGS. 50-53 show details of the rails. FIG. 50 shows the upper rail and the locking mechanism which accepts the cylinder, while FIG. 51 shows the connection to the wall stud on the rear (to the right) and the locking mechanism to the front (left). The lower rail shown in FIGS. 52 and 53 has a cup into which the cylinder slides and may include connections for power or data: the power cord in FIG. 53 is shown leaving from the connection to the wall stud on the right-hand side of the figure.

FIGS. 54a and 54b are top and side views similar to FIG. 14 but with a different arrangement of coatings on a layer. Thus the outer areas are antibacterial compounds, as are the inner areas. However, a narrow stripe running in a track around the surface of the device and furthermore connected to the peel tabs is adhesive compound, thus allowing easy removal and yet a majority of the surface to be antibacterial.

FIG. 55 is a side view showing that the swing arm can fold in on the cylinder and FIG. 56 is a side view showing the same. This particular fold, examined carefully, is different from the fold previously displayed: the dimension is different. This increases the convenience of the device by allowing it to fold entirely away when not needed, for example, right up against a wall.

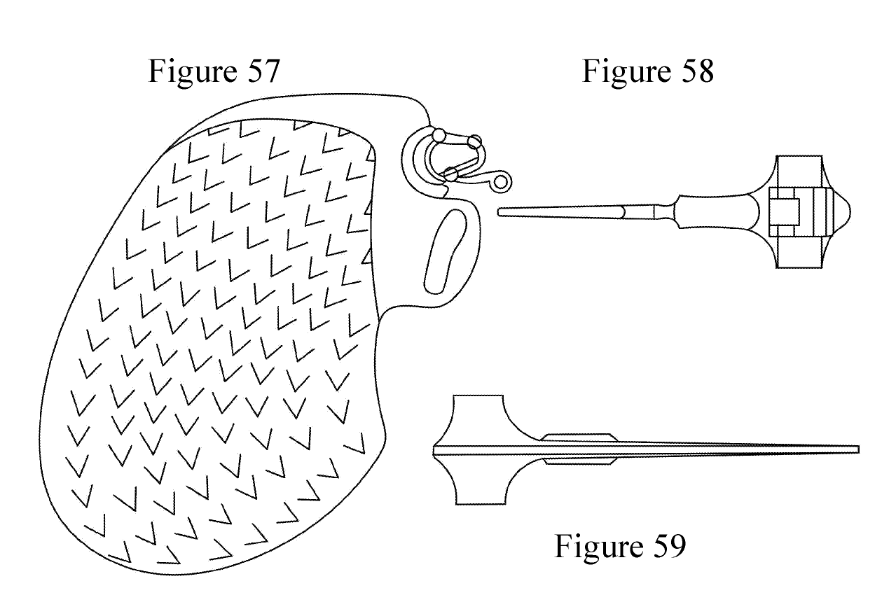

FIG. 57 is a top view of an embodiment having additional friction control devices (raised areas) thereon. The “V” shaped raised areas allow an easy insertion of the device between a mattress and box spring or between a hospital mattress and the hospital bed surface, but then provide considerably more friction against retraction, thus securing it in place. The handle is obviously necessary for convenience, as is the locking mechanism designed for a mechanical engagement to the cylinder or support. Note that the planform (shape) of the paddle is unique, and is specifically designed for an easy “rotation insertion” into the mattress: this also coordinates with the raised “V” shapes, which outline the curvilinear insertion motion used.

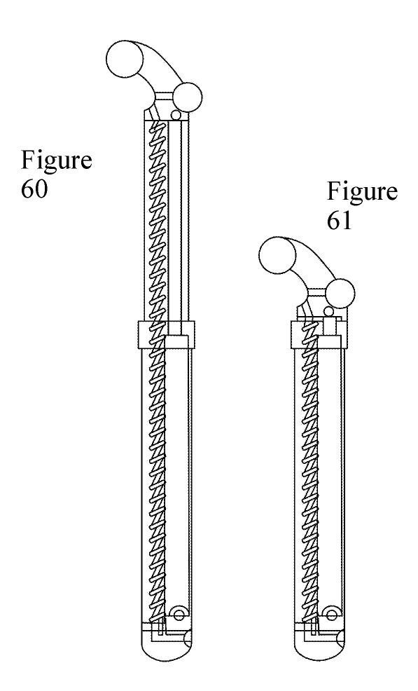

FIG. 58 is a side view of the mattress insert support, and FIG. 59 is also a side view of the mattress insert support, showing the angled shape for easy installation and the raised support for the cylinder/arm/other support: this raised or sleeved area provides greater mechanical strength.

FIG. 60 is a side view in partial cutaway of a strut of the invention and FIG. 61 is a side view in partial cutaway of a strut of the invention: the first in the extended position and the second retracted. It may be seen that the knuckle at the top allows attachment to the swing arm, and the internal flexible cord (coiled form optional) allows a power or electronic connection such as a USB cable, telephone, or 120 VAC. The knob at the top knuckle allows tightening and loosening to control the motion. The actuator inside the strut may be powered, gas, hydraulic, electrical, etc.

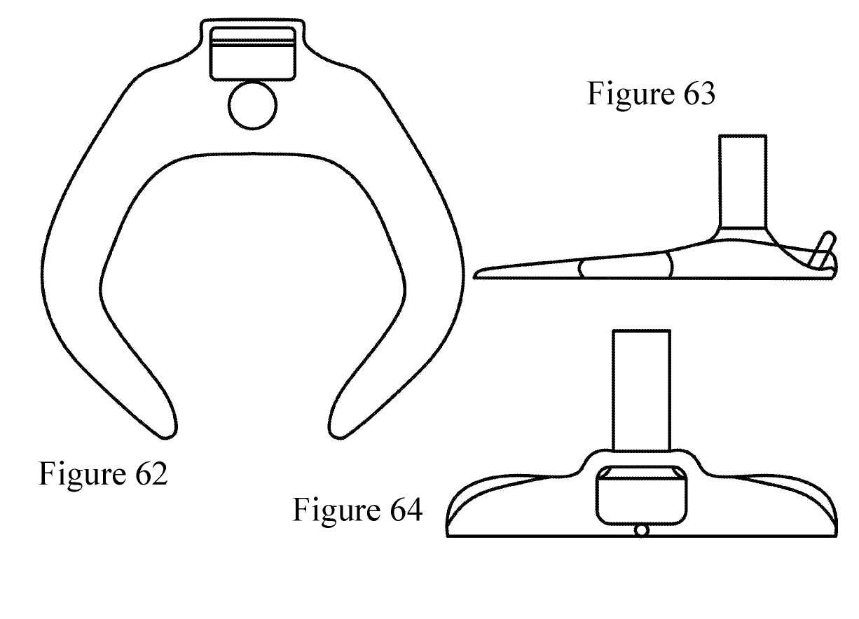

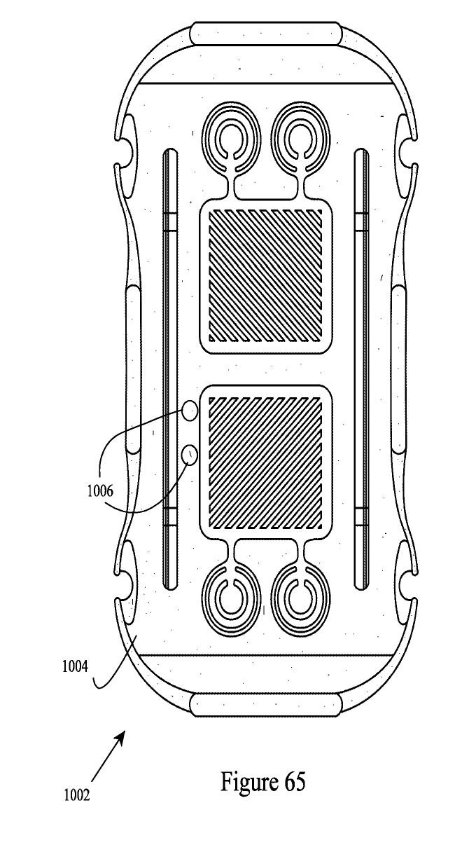

FIG. 62 is a top view of the claw stand of the invention in another embodiment, FIG. 63 is a side view of the claw stand of the invention, while FIG. 64 is a rear view of the claw stand of the invention. The unique shape allows it to easily slide around other objects, such as table legs, bed legs, medical equipment stands and so on. A handhold or handle seen on the rear allows easy motion. It is furthermore wedge shaped (see side view 63) to allow insertion under other structures and has a power connection (bottom of rear view).

The claw stand may be quite heavy in optional embodiments: this provides an advantageous stability, at the cost of making it harder for more lightly built individuals to move.

In addition to the features and aspects, advantages and embodiments previously described, the present invention has the following elements.

The shape of the tray unique design has concords in a frame supporting multiple layers of thin film.

The tray frames are shaped to support the function of holding items. The tray frame's bottom is designed to mechanically slide into a support frame. The tray frame will be made of disposable or non-disposable materials. The tray frame can connect to power or data once attached to support frame. The tray frame will support monitoring sensors and lighting. The tray frame is designed to connect to a support frame at multiple angles above and below horizontal. The tray frame is designed to hold personal electronics (laptop computer, tablet computer and personal phones) of multiple sizes.

The tray frame is designed to allow liquid to flow to the center. The tray frame can be injected with antibacterial, fire retardant and or other compounds. The tray frame is designed to support handles to move the tray.

The films unique layering may be of compounds between chemically injected layers of film.

The film can be removed by pinching tabs then pulling towards the center of tray. The film tabs are located around tray frame edges at the bottom. The film layers are attach by adhesives, electrostatic and or ultrasonic welding. The film can have raised ridges to separate compounds applied. The film can have liquid conductivity compounds that feed data between forks or prongs. The film can contain properties of compounds to detect bacteria growth by changing colors. The film can contain lines of conductive adhesives compound that can be attached to sensors that will kill bacteria and or transfer information. The film can contain lines of design such as logo and become visual or illuminate with tray frame lighting.

The film layering can be multiple layers and supports its own structure molded to fit the tray frame. The film layering can be multiple layers, not supporting its own structure and mold to fit existing surface. The film can come in packet layers and replaced with refill connecting in tray frame. The film layers can contain printing such as advertisements, instructions and the like.

The film is flexible to allow to be used like a drop cloth. The film can have electronically charged neg. or pos. ions that can effect the surface of each individual layer or all layers. The films electronically charged surface will make the connection through the tray frame then thru the fork connector

The film can have a gel type coating compound that dissolves over a timed period. When gel type coating is dissolved a word like (peel layer) can become visible.

The lighting in the fork design can flash UV Pulse through the clear layers of film killing bacteria. The tray frame and films can be different in colors or in patterns to direct UV Pulse light beam to high contact areas.

The tray frame and film can contain its own power source and or processor for data information collection.

The fork is designed to except the tray frame and hold in place with click connect on the inside of the fork rail with tension. The click connection on the inter fork can be a point or source of low voltage feed and or data to the tray frame and peel away film. The lower part of the fork is designed to accept an external connection to support a net, drawer, speaker, camera, trash bag, etc. The fork on either side is hinged to allow the prong to move individually above or below 0 degrees to support a flat tray or angled stair step designed tray frame. The fork prong can contain lighting to illuminate the tray frame and or film. This lighting can be for illumination or killing of bacteria with UV Lighting. The fork power and data source will connect at the engagement of the fork swivel locking connector.

The fork can be designed to provide sensors internal or external to detect such things as weight placed on fork, motion, temperature beam, and or bacteria. It may have Bluetooth or wi-fi for data to/from a network directly to a smartphone, tablet or computer.

The fork can be made with impregnated anti-bacteria properties in plastic, metals, copper etc.

The fork can accept attachments to reconfigure its structure as needed. The fork is designed to be pinched by the tray frame film cartage. The fork can also change it configuration to support different designs of tray frames.

The connector locks the fork into place. The connector connects power to the fork/prongs. The connector is unique in shape to lock fork/prong into and the connector can be tightened to adjust motion of swings of various parts. The connector has lighting, buttons, sensors and or processor for data. The connector can have 360 degrees motion. The connector locks fork/prong with a locking mechanism. The connector powers and or data feeds the fork/prong through frame into film.

The arm contains controls for horizontal motion and functions in tray/prong through to film. The arm contains USB ports for charging and data feed and can contain wire, cable, tubing to support power and or data feed. The arm can contain support wiring within the arm structure and or attached external wire underneath arm and can support extra external wiring in storage area underneath arm and held in place with rubber flaps across from each other overlapping to support.

The arm frame can be made of metal, plastic, layered composite materials.

Note that the arm can be fitted with touch surfaces film peel a ways used with the same protection as film layers, can have a cover over it made of anti-bacterial plastic, copper and or peel-away layers of film, and the arm's unique design at the controls is designed to fit the palm of a user's hand comfortably allowing a finger forward to press buttons. The arm's swinging motion can be adjusted by tightening knob where control arm connects to support arm. The arm's unique design located on top is designed not to pinch fingers when folded on top of the support arm.

The arm can contain wire, cable, tubing to support power and or data feeds, and can contain support wiring within the arm structure and or attached external wire underneath arm.

The arm can support extra external wiring in storage area underneath arm and held in place with rubber flaps across from each other overlapping to support. The arm frame can be made of metal, plastic, layered composite materials.

The arm can be fitted with touch surfaces film peel a ways used with the same protection as film layers. The arm can except a cover over it made of anti-bacterial plastic, copper and or peel away layers of film.

The arm's swinging motion can be adjusted by tightening knob where support arm connects to knuckle.

The support arm can fold inward to the cylinder unit and can fold from 0 degrees to 90 degrees.

The support arm also locks in place at knuckle at needed degrees, much like the main arm discussed previously. The support arm can be disengaged by mechanical button. Support arm's unique design located on top is designed not to pinch fingers when folded below the control arm.

The knuckle connects to the support arm. The knuckle can turn 360 degrees and the knuckle swing motion can be tightened with knob. The knuckle is located at the top of the actuator cylinder. The knuckle supplies power and or data to the support arm at connection. The knuckle and or knob can be covered over with materials made of anti-bacterial plastic, copper and or peel away layer of film as other parts of the furniture.

The knuckle is directly attached to the top of the cylinder. The cylinder is telescopic so it can move up or down. The cylinder may be uniquely designed to fit proprietary cylinder mounts, and the cylinder can be shaped round, square, triangle, egg and or and other shape. The cylinder mechanical movement can be supplied by gas, electric and or hydraulic actuators.

The cylinder can supply power and or data through to the fork and or prongs and can supply power to the top at all times while in motion.

At cylinder's telescopic joint an antibacterial donut can be place to sterilize the upper tube as it moves up or down. The cylinder power and or data connection is located at the bottom of the cylinder. The cylinder bottom ball is rubber or other materials to secure to all flooring surfaces, wood, tile, carpet and or any other flat surface. Cylinder tubing can be made of metal, carbon fiber, fiberglass, plastic and or other materials. The cylinder can have lighting to notify users that power is connected or no power is running through the cylinder.

The cylinder is designed to attach to a floor mount, mattress paddle mount and or a track wall mount device.

Floor claw mount is designed to accept the cylinder to secure it during use. The floor claw mount is uniquely designed to slide between a support leg of a chair, couch and or bed post between a wall easily pushed into position by guiding between the two obstacles and curving its self into place.

The floor claw mount is uniquely designed at a slope higher at the cylinder mount and lower at the tips to allow the mount to wedge under a chair, couch, and or bed to add support. The floor claw mount is uniquely designed to allow itself to lock with a wedge and pin technique then unlocking by grabbing the handle pulling and twisting allowing the claw mount slide out of its wedge formed lock.

The floor claw mount provides power and or data to the cylinder as a click-in connection once placed in to cylinder shelve. The floor claw mount provides external power and or data connection that provides the power and or data feed to the bottom of the cylinder sleeve. The floor claw mount can hold data processors for the functions of the whole connected unit.

The floor claw mount can be made of metal, concrete, plastic and or cast molded. The floor claw mount can be molded by casting, injection molding and or spin molding.

The floor claw mount can have attachments on the bottom of it allowing easier sliding on surfaces and or wheels to roll across surface. The paddle's unique design allows it to slide between the mattress and box spring then supporting cylinder by a mechanical connection.

The paddle's kidney shape allows it to be pushed into place by sliding the tip between the mattress and box spring the curved shape guides the paddle into position by the curve touching the back board and or wall then sliding to position. The paddle has a support on either side extending from the paddle to support the cylinder.

The paddle can be made of metal, plastic, wood and or composite materials. The paddle can have raised patterns on both sides or one surface allowing the paddle to secure its self to the fabric. The paddle has a handle used to insert and or remove from the mattress and box spring. The paddle can be use on either side of the bed right side and or left side by flipping the paddle over.

The paddle can also be used as a table when cylinder is connected to the floor claw mount.

The rail mount has two rails upper rail and lower rail. Rail mounts can be 2, 4, and 6 feet long and or can be attached to continue all the way down a wall horizontally.

The rail mounts allow the cylinder to move horizontally then lock into place where needed and unlocked to move to new location. The upper rail has a mechanical lock device that locks the cylinder into place once the cylinder connected into the lower rail. The upper rail has two caps at either end capping the end openings of the rail. The rails be mount to a wall or mount off the wall with spacers allowing electric cord to slide between the wall and rail. The lower rail will contain a power and or data strip inside the rail to make connect to the cylinder once placed into sleeve cup like holder. The lower rail has two caps at either end capping the end openings of the rail with one of the caps supplying the power and or data. The cylinder slides into a cup shaped sleeve to make power and or data connection for the whole unit on the lower rail. The rails can be made of metal, plastic and or composite materials.

FIG. 65 is atop view of a tray of the invention having perforations in the topmost visible layer. Tray 1002 has film 1004 which is not solid, rather it has numerous very small pores 1006 which allow liquid or moisture to pass through this layer of film. In general these are referred to herein as “capillaries” or capillary pores, regardless of whether the size is appropriate for capillary action or not.

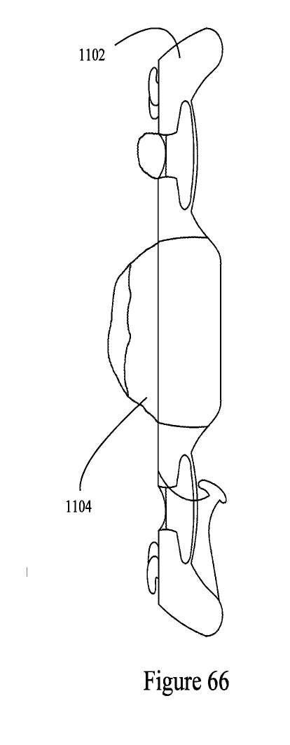

FIG. 66 is a side view of a tray of the invention having bags passing through some of the holes. Tray 1102 has a portion which does not have the backing/structural support layer, but which does have the flexible sanitary/hygienic layers. These layers have extra material which hangs below the tray, forming a bag-like pocket 1104 below the tray. Thus a void space in the tray is defined by the hole through the backing layer, and the bag-like pocket hanging down is defined by the extra material of the flexible hygienic layer.

Bag-like pocket 1104 may be seen to be flexible, hygienic and water-tight, since the layers are the same layers on the tray top surface, without any breaks or seams. The bag may be thought of as simply an extension of the normal hygienic layers which form the bottoms of the cup holders, tablet holders and so on in the various depressions in the tray.

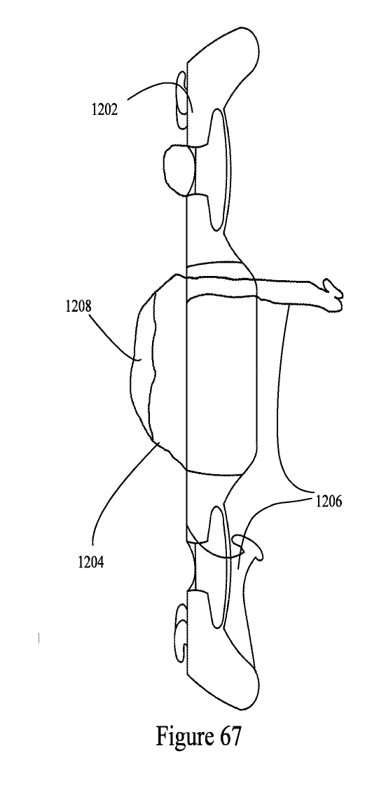

FIG. 67 is a side view of a tray of the invention half way through the process of having a film removed so as to form a bag for liquids at the lowest point in the tray. Tray 1202 has bag 1204 still not removed. It hangs below the tray as previously shown, and it may be seen that it is the lowest point in the tray. As seen previously, the tray surface may be formed so that liquid spilt upon the tray surface may freely flow through shallow channels atop the tray and into the bag 1204, where it collects (1208).

Removed parts 1208 are portions of the film which have been lifted off the tray surface. By peeling upward from the edge of the tray, the user has allowed the bag-like pocket 1204 to remain in place as the film/walls of the bag rise higher. When the bag is finally removed, the liquid remains safely contained therein.

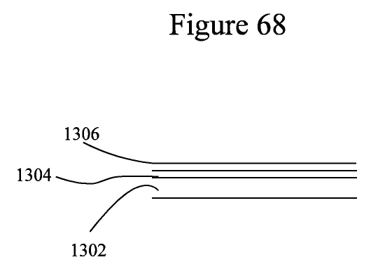

FIG. 68 is a side cross-sectional view of one exemplary arrangement of layers of the invention having two or more coatings per layer. Solid film 1302 has more than one coat thereon. This plurality of coats allows for the easy use of more than one type coating: multiple antibiotics, antifungals, adhesives, microbial detector coatings and so on and so forth.

First coat 1304 has above it a second coat 1306 with no intervening film layer.

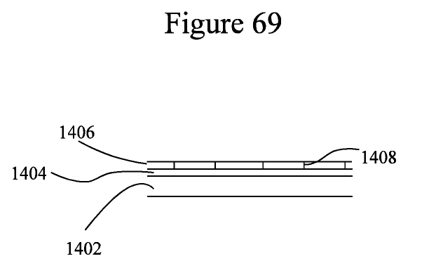

FIG. 69 is a side cross-sectional view of one exemplary arrangement of layers of the invention having perforations with an absorbing layer beneath. Solid film 1402 carries a spongy layer 1404 which in turn has a perforated layer 1406 having multiple pores 1408 as shown previously in FIG. 65. In use, a liquid spilt upon the top surface of the tray may be absorbed through the pored layer 1406 and into the spongy layer 1404.

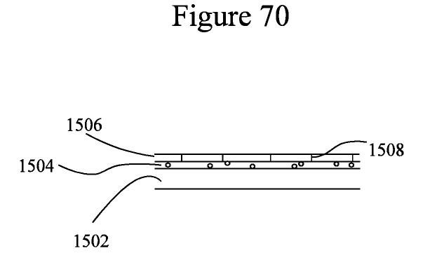

Liquid flow may advantageously be the opposite direction if the liquid is anti-microbial, or any liquid having beneficial properties. FIG. 70 is a side cross-sectional view of one exemplary arrangement of layers of the invention having perforations with a liquid antimicrobial compound able to wick, flow or flow by capillary action upward through the perforated layer above. Solid layer 1502 has a liquid antimicrobial coating 1504 thereon, which may be a gel, a true liquid, a paste, very small particles or otherwise able to pass through pores 1508 in the perforated layer 1506. The antimicrobial or other liquid may rise through wicking, capillary action, by pressure applied to the tray surface, evaporation and so on and so forth. The frame tank media (the tank and the liquid, or one or the other) may conduct electricity in embodiments. It may also contain beneficial microorganisms, and may be illuminated from below, from the side, or from above. It may be transparent to allow light to pass through.

One advantage of the use of liquids is that the liquid may be chosen for its ability to detect bacteria or other microorganisms.

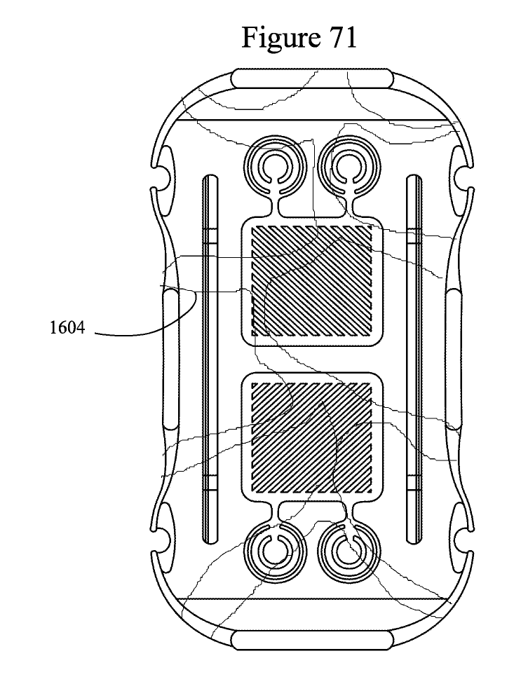

FIG. 71 is a top view of a tray having the coating applied in a spider web pattern. Spider web pattern coating 1604 might be employed to save material or for other reasons. The spider web pattern shown is obviously merely exemplary. The compounds on the coatings may “bleed” from the strands of web onto the open areas to be protected.

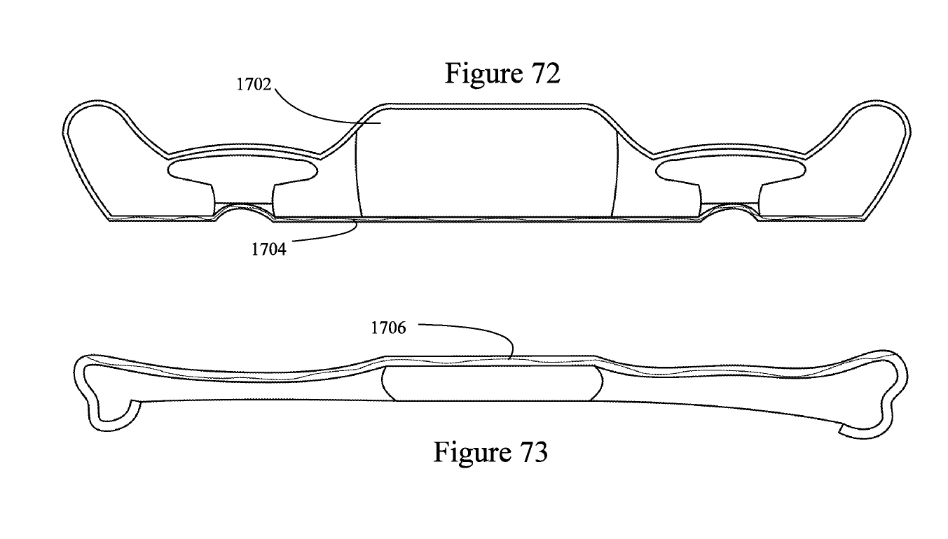

FIG. 72 is a side view of an embodiment in which the tray of the device is also a liquid storage reservoir or tank for the antimicrobial compounds.

Tray 1702 have a tank (with liquid shown inside the tank) which is a thin shell, for example tank/shell 1704. This embodiment might be used with the pores of the previous diagrams or with similar arrangements.

FIG. 73 is a top view of an embodiment in which the tray of the device is also a liquid storage reservoir for the antimicrobial compounds. The tray is differently shaped but does have tank/shell 1706.

Methods and components are described herein. However, methods and components similar or equivalent to those described herein can be also used to obtain variations of the present invention. The materials, articles, components, methods, and examples are illustrative only and not intended to be limiting.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art.

Having illustrated and described the principles of the invention in exemplary embodiments, it should be apparent to those skilled in the art that the described examples are illustrative embodiments and can be modified in arrangement and detail without departing from such principles. Techniques from any of the examples can be incorporated into one or more of any of the other examples. It is intended that the specification and examples be considered as exemplary only, with a true scope and spirit of the invention being indicated by the following claims.

I claim:

1. A disposable sanitary tray comprising:
a backing, the backing being made of a resilient material able to bear weight, the backing formed into a tray shape having an edge and having a top surface;
a plurality of hygienic layers, each hygienic layer comprising a film and a coating, each coating being one member selected from the group consisting of: anti-microbial coatings, anti-bacterial coatings, bacterial detection coatings, adhesives, and combinations thereof.

2. The disposable sanitary tray of claim 1, further comprising: at least one depression, the depression defined by the backing and conformed to by the plurality of hygienic layers, the at least one depression dimensioned and configured to accept one edge of a personal electronic device.

3. The disposable sanitary tray of claim 1, further comprising: at least one depression, the depression having an opening defined by a void of the backing, with a bag-like pocket passing through the voice of the backing, the bag-like pocket defined by the plurality of hygienic layers.

4. The disposable sanitary tray of claim 1 wherein the backing and the hygienic layers are the same, the hygienic layers offering the tray structural support.

5. The disposable sanitary tray of claim 3, for use with a liquid such as spilled beverages, bodily fluids or the like, wherein the tray has a lowest point, the lowest point being within one or more of the bag-like pockets,
the tray being dimensioned to allow such liquid to flow across at least a portion of the tray to the lowest point within one or more bag-like pockets,
whereby such liquid on the surface of the tray may be disposed of by peeling a single hygienic layer from the tray edge first, so that such liquid remains contained within one or more bag-like pockets while the single hygienic layer is removed from the tray.

6. The disposable sanitary tray of claim 1, for use with a liquid such as spilled beverages, bodily fluids or the like, wherein:
at least a first one of the hygienic layers is perforated so as to allow such liquid to pass through, and at least a second one of the hygienic layers is directly underneath the first hygienic layer and is comprised of an absorbent material.

7. The disposable sanitary tray of claim 1, further comprising:
a first capillary layer perforated by capillary passages;
a second antimicrobial layer impregnated with an antimicrobial liquid, whereby the antimicrobial liquid may pass upward through the first capillary layer to the surface.

8. The disposable sanitary tray of claim 1, wherein the film has antimicrobial properties and the coating is omitted.

9. The disposable sanitary tray of claim 1, wherein the film comprises a material which is one member selected from the group consisting of: polymer films, metallized polymer films, metal foils, paper, cloth and combinations thereof.

10. The disposable sanitary tray of claim 1, further comprising:
a second coating selected from the group consisting of: anti-microbial coatings, anti-bacterial coatings, microbial detection coatings, bacterial detection coatings, adhesives, and combinations thereof.

11. The disposable sanitary tray of claim 1, wherein the anti-microbial coating comprises one member selected from the group consisting of: antibiotic compounds, essential oils, copper, metals, disinfectants, sanitizers, bactericidal agents, bacteriostatic agents, antifungal agents, antiviral agents, antiparasitic agents, liquid agents, liquids having current passing therethrough, liquids having light passing therethrough, beneficial micoroorganisms, and combinations thereof.

12. The disposable sanitary tray of claim 1, further comprising:
a reservoir having therein a liquid antimicrobial coating.

13. The disposable sanitary tray of claim 12 wherein the liquid antimicrobial coating further has current passing therethrough, light passing therethrough, UV light passing therethrough, beneficial microorganisms and combinations thereof.

14. The disposable sanitary tray of claim 1, wherein the antimicrobial coating covers a first portion of the tray top surface, the first portion being one member selected from the group consisting of: a spider web of thin lines, less than half of the top surface, more than half of the top surface, all of the top surface.

15. A bedside table comprising:
a core unit having at least one arm segment, the at least one arm segment dimensioned and configured to project over such bedside;
a connector unit mounted upon the core unit at a first end of the at least one arm segment;
a backing, the backing being made of a resilient material able to bear weight, the backing formed into a tray shape, the tray shape dimensioned and configured to mechanically engage such connector unit;
a plurality of hygienic layers, each hygienic layer comprising a film and a coating, each coating being one member selected from the group consisting of: anti-bacterial coatings, bacterial detection coatings, adhesives, and combinations thereof.

16. The bedside table of claim 15, further comprising:
an electronic connector suitable for connecting an electronic device to.

17. The bedside table of claim 16, wherein the connector unit is a pair of tines and the electronic connector unit is one member selected from the group consisting of: an electrical power cord, an electronic data cable, electrical controls, low voltage connectors suitable for passing low voltage through the films, UV lighting, ambient lighting, and combinations thereof.

18. The bedside table of claim 16, further comprising: low voltage conductivity through the compounds of/in the film.

19. The bedside table of claim 15, wherein the hygienic layers are transparent in UV and wherein the table further comprises: a UV light disposed so as to shine through the tray.

20. The bedside table of claim 15, for use with a floor or a mattress and further comprising:
a foot, the foot having a shape selected from the group consisting of: a foot dimensioned and configured to support the table when the foot is flat on such floor, a foot dimensioned and configured to support the table when the foot is inserted beneath such mattress and combinations thereof.
